(19) United States
(12) Patent Application Publication
(10) Pub. No.: US 2015/0010584 A1
(43) Pub. Date: Jan. 8, 2015

(54) Targeted Delivery Of Autoantigens To B Cell Populations

(71) Applicant: The University of Massachusetts, Boston, MA (US)

(72) Inventors: Ann Marshak-Rothstein, Newton, MA (US); Krishna Moody, Worcestar, MA (US)

(21) Appl. No.: 14/315,557

(22) Filed: Jun. 26, 2014

Related U.S. Application Data
(60) Provisional application No. 61/842,703, filed on Jul. 3, 2013.

(51) Int. Cl. A61K 47/48; C07K 14/77; G01N 33/564; C07K 16/42

(52) U.S. Cl. CPC A61K 47/4833; C07K 16/42; C07K 14/77; G01N 33/564; G01N 2800/24; G01N 2800/065; G01N 2800/042; G01N 2800/101; G01N 2800/285
USPC 424/178.1; 530/367; 435/69.6; 436/501; 435/7.6; 435/7.92

(57) ABSTRACT

The present invention is related to compositions and methods to stimulate the immune system. For example, antigen-specific antibodies may be produced by stimulating B cell populations with specific antigenic compounds, such as an adjuvant comprising a macromolecule capable of activating a Toll-Like receptor (TLR). For example, a BCR adapter IgM (BCRAM) is described to exemplify delivery of autoantigens to polyclonal B cell populations resulting in immunoactivation by TLR activation. Alternatively, a compound is described that inhibits TLR activation.

### STATEMENT OF GOVERNMENT INTEREST

This invention was made with government support awarded by the National Institutes of Health (grant number NIH AR 05025). The government has certain rights in the invention.

### FIELD OF THE INVENTION

The present invention is related to compositions and methods to stimulate the immune system. For example, antigen-specific antibodies may be produced by stimulating B cell populations with specific antigenic compounds, such as an adjuvant comprising a macromolecule capable of activating a Toll-Like receptor (TLR). For example, a BCR adapter IgM (BCRAM) is described to exemplify delivery of autoantigens to polyclonal B cell populations resulting in immunoactivation by TLR activation. Alternatively, a compound is described that inhibits TLR activation.

### BACKGROUND

Systemic lupus erythematosus (SLE)4 and other autoimmune diseases are characterized by the development of autoantibodies directed against a limited subset of nucleic acid-containing autoantigens including, but not limited to, DNA, chromatin, and ribonucleoproteins. Plotz, P. H. 2003 “The autoantibody repertoire: searching for order” Nat. Rev. Immunol. 3:73-78. Defects in the clearance of apoptotic material have been associated with the development of anti-nuclear antibodies and autoimmune disease. Walport, M. J. 2000 Lupus “DNase, and defective disposal of cellular debris” Nat. Genet. 25:135-136.

However, the mechanism(s) leading to the production of DNA-reactive autoantibodies is difficult to explain since mammalian DNA is a poor immunogen compared with microbial DNA. Messina et al., 1991 “Stimulation of in vitro murine lymphocyte proliferation by bacterial DNA” J. Immunol. 147: 1759-1764; Klinman et al., 1996 “CpG motifs present in bacteria DNA rapidly induce lymphocytes to secrete interleukin 6, interleukin 12, and interferon gamma” Proc. Natl. Acad. Sci. USA 93:2879-2883; and Yamamoto et al., 1992 “DNA from bacteria, but not from vertebrates, induces interferons, activates natural killer cells and inhibits tumor growth” Microbiol. Immunol. 36: 983-997. It has been reported that immune complexes (ICs) containing mammalian DNA can activate IgG-autoreactive B cells through a mechanism dependent on engagement of the BCR and the intracellular pattern-recognition receptor TLR9. Leadbetter et al. 2002 “Chromatin-IgG complexes activate B cells by dual engagement of IgM and Toll-like receptors” Nature 416:603-607.

What is needed in the art is a suitable technology for modeling autoantigen activation of B cells to research and treat the development and progression of autoimmune diseases, such as systemic lupus erythematosus (SLE), by identifying a suitable drug target.

### SUMMARY OF THE INVENTION

The present invention is related to compositions and methods to stimulate the immune system. For example, antigen-specific antibodies may be produced by stimulating B cell populations with specific antigenic compounds, such as an adjuvant comprising a macromolecule capable of activating a Toll-Like receptor (TLR). For example, a BCR adapter IgM (BCRAM) is described to exemplify delivery of autoantigens to polyclonal B cell populations resulting in immunoactivation by TLR activation. Alternatively, a compound is described that inhibits TLR activation.

In one embodiment, the present invention contemplates a B Cell Receptor Adapter Immunoglobulin M (BCRAM) complex comprising an IgG variable domain linked to an ovalbumin fragment, wherein the ovalbumin fragment is linked to an acceptor peptide, wherein the acceptor peptide is linked to an IgG Fe-binding domain. In one embodiment, the IgG variable domain has specific affinity for an α-IgM antibody. In one embodiment, the α-IgM antibody is a B cell receptor. In one embodiment, the IgG variable domain has specific affinity for a murine α-IgM antibody. In one embodiment, the murine α-IgM antibody is a murine B cell receptor. In one embodiment, the IgG variable domain has specific affinity for a human α-IgM antibody. In one embodiment, the human α-IgM antibody is a human B cell receptor. In one embodiment, the acceptor site comprises a biotin molecule. In one embodiment, the IgG Fe binding domain binds to an autoantibody. In one embodiment, the IgG Fc binding domain includes, but is not limited to, Protein A and/or Protein G. In one embodiment, the autoantibody binds to an autoantigen. In one embodiment, the autoantigen is an RNA. In one embodiment, the autoantigen is DNA. In one embodiment, the autoantigen is α-chromatin. In one embodiment, the autoantigen is a synthetic autoantigen. In one embodiment, the autoantigen is a derivatized autoantigen. In one embodiment, the autoantigen is a conjugated autoantigen. In one embodiment, the autoantigen is an autoimmune disease autoantigen. In one embodiment, the autoimmune disease autoantigen includes, but is not limited to, a type 1 diabetes autoantigen, an alopecia areata autoantigen, a systemic lupus erythematosus autoantigen, a Behçet's disease autoantigen, a Sjögren's syndrome autoantigen, a rheumatoid arthritis autoantigen, a Grave's disease autoantigen, an antiphospholipid antibody syndrome autoantigen, a multiple sclerosis autoantigen, an irritable bowel disease autoantigen, a Crohn's disease autoantigen, an ulcerative colitis autoantigen and/or a dermatomyositis autoantigen.

In one embodiment, the present invention contemplates a method for producing autoantigen-specific antibodies comprising; a) providing; i) a B Cell Receptor Adaptor Immunoglobulin M (BCRAM) complex, ii) an autoantibody-autoantigen complex, and iv) a cell culture wherein at least one cell comprises a B cell receptor (BCR) and a Toll-Like receptor (TLR); b) binding the autoantibody-autoantigen complex to the BCRAM to form a BCRAM-autoantibody-autoantigen complex; c) targeting the BCRAM-autoantibody-autoantigen complex to the BCR to form an internalized BCRAM-autoantibody-autoantigen/BCR complex within said at least one cell; and d) activating the TLR with the internalized BCRAM-autoantibody-autoantigen/BCR complex, wherein autoantigen-specific antibodies are generated. In one embodiment, the TLR is TLR7. In one embodiment, the TLR is TLR9. In one embodiment, the TLR is TLR-3. In one embodiment, the TLR is TLR8. In one embodiment, the TLR is located in an intracellular compartment. In one embodiment, the autoantigen is a synthetic autoantigen. In one embodiment, the autoantigen is a derivatized autoantigen. In one embodiment, the autoantigen is a conjugated autoantigen. In one embodiment, the autoantigen is an autoimmune disease autoantigen. In one embodiment, the autoimmune disease autoantigen includes, but is not limited to, a type 1 diabetes autoantigen, an alopecia areata autoantigen, a systemic lupus erythematosus autoantigen, a Behçet's disease autoantigen, a Sjögren's syndrome autoantigen, a rheumatoid arthritis autoantigen, a Grave's disease autoantigen, an antiphospholipid antibody syndrome autoantigen, a multiple sclerosis autoantigen, an irritable bowel disease autoantigen, a Crohn's disease autoantigen, an ulcerative colitis autoantigen and/or a dermatomyositis autoantigen.

In one embodiment, the present invention contemplates a method for detecting autoimmune disease autoantibodies comprising: a) providing; i) a B Cell Receptor Adaptor Immunoglobulin M (BCRAM) complex comprising an autoantibody-autoantigen complex, wherein the autoantibody-autoantigen complex has specific affinity for an autoimmune disease antibody; iii) a biological sample derived from a patient, wherein said sample is suspected of comprising the autoimmune disease antibody; b) contacting the BCRAM complex with the biological sample under conditions such that the autoimmune disease antibody is detected. In one embodiment, the autoimmune disease antibody is detected before treatment for the autoimmune disease begins. In one embodiment, the autoimmune disease antibody is detected after treatment for the autoimmune disease begins. In one embodiment, the detection of the autoimmune disease antibody diagnoses an autoimmune disease. In one embodiment, the autoantigen is a synthetic autoantigen. In one embodiment, the autoantigen is a derivatized autoantigen. In one embodiment, the autoantigen is a conjugated autoantigen. In one embodiment, the autoantigen is an autoimmune disease autoantigen. In one embodiment, the autoimmune disease autoantigen includes, but is not limited to, a type 1 diabetes autoantigen, an alopecia areata autoantigen, a systemic lupus erythematosus autoantigen, a Behçet's disease autoantigen, a Sjögren's syndrome autoantigen, a rheumatoid arthritis autoantigen, a Grave's disease autoantigen, an antiphospholipid antibody syndrome autoantigen, a multiple sclerosis autoantigen, an irritable bowel disease autoantigen, a Crohn's disease autoantigen, an ulcerative colitis autoantigen and/or a dermatomyositis autoantigen.

In one embodiment, the present invention contemplates a method for treating an autoimmune disease comprising: a) providing; i) a B Cell Receptor Adaptor Immunoglobulin M (BCRAM) complex comprising an autoantibody-autoantigen complex; ii) a cell comprising a B cell receptor (BCR) and a Toll-Like receptor (TLR), wherein the cell is within a patient exhibiting at least one symptom of an autoimmune disease; b) administering the BCRAM complex to the patient under conditions such that the at least one symptom is reduced. In one embodiment, the BCRAM complex comprises a human IgG variable domain. In one embodiment, the TLR is TLR7. In one embodiment, the TLR is TLR9. In one embodiment, the TLR is TLR-3. In one embodiment, the TLR is TLR8. In one embodiment, the TLR is located in an intracellular compartment. In one embodiment, the autoantigen is a synthetic autoantigen. In one embodiment, the autoantigen is a derivatized autoantigen. In one embodiment, the autoantigen is a conjugated autoantigen. In one embodiment, the autoantigen is an autoimmune disease autoantigen. In one embodiment, the autoimmune disease autoantigen includes, but is not limited to, a type 1 diabetes autoantigen, an alopecia areata autoantigen, a systemic lupus erythematosus autoantigen, a Behçet's disease autoantigen, a Sjögren's syndrome autoantigen, a rheumatoid arthritis autoantigen, a Grave's disease autoantigen, an antiphospholipid antibody syndrome autoantigen, a multiple sclerosis autoantigen, an irritable bowel disease autoantigen, a Crohn's disease autoantigen, an ulcerative colitis autoantigen and/or a dermatomyositis autoantigen.

### DEFINITIONS

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis.

The term “at risk for” as used herein, refers to a medical condition or set of medical conditions (e.g., risk factors) exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “linked” or “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “derived from” as used herein, refers to the source of a compound or sequence. In one respect, a compound or sequence may be derived from an organism or particular species. In another respect, a compound or sequence may be derived from a larger complex or sequence.

The term “test compound” as used herein, refers to any compound or molecule considered a candidate as an inhibitory compound.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “polypeptide”, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens or larger.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term, “purified” or “isolated”, as used herein, may refer to a peptide composition that has been subjected to treatment (i.e., for example, fractionation) to remove various other components, and which composition substantially retains its expressed biological activity.

Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that some trace impurities may remain.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

The terms “amino acid sequence” and “polypeptide sequence” as used herein, are interchangeable and to refer to a sequence of amino acids.

As used herein the term “portion” when in reference to a protein (as in “a portion of a given protein”) refers to fragments of that protein. The fragments may range in size from four amino acid residues to the entire amino acid sequence minus one amino acid.

The term “portion” when used in reference to a nucleotide sequence refers to fragments of that nucleotide sequence. The fragments may range in size from 5 nucleotide residues to the entire nucleotide sequence minus one nucleic acid residue.

The term “antibody” refers to immunoglobulin evoked in animals by an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

The terms “specific binding” or “specifically binding” when used in reference to the interaction of an antibody and a protein or peptide means that the interaction is dependent upon the presence of a particular structure (i.e., for example, an antigenic determinant or epitope) on a protein; in other words an antibody is recognizing and binding to a specific protein structure rather than to proteins in general. For example, if an antibody is specific for epitope “A”, the presence of a protein containing epitope A (or free, unlabeled A) in a reaction containing labeled “A” and the antibody will reduce the amount of labeled A bound to the antibody.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The term “derivative” as used herein, refers to any chemical modification of a nucleic acid or an amino acid. Illustrative of such modifications would be replacement of hydrogen by an alkyl, acyl, or amino group. For example, a nucleic acid derivative would encode a polypeptide which retains essential biological characteristics.

The term “biologically active” refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells which lack protein activity with an expression vector which expresses the protein, a derivative thereof, or a portion thereof.

The term “immunologically active” defines the capability of a natural, recombinant or synthetic peptide, or any oligopeptide thereof, to induce a specific immune response in appropriate animals or cells and/or to bind with specific antibodies.

The term “antigenic determinant” as used herein refers to that portion of a molecule that is recognized by a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The terms “immunogen,” “antigen,” “immunogenic” and “antigenic” refer to any substance capable of generating antibodies when introduced into an animal. By definition, an immunogen must contain at least one epitope (the specific biochemical unit capable of causing an immune response), and generally contains many more. Proteins are most frequently used as immunogens, but lipid and nucleic acid moieties complexed with proteins may also act as immunogens. The latter complexes are often useful when smaller molecules with few epitopes do not stimulate a satisfactory immune response by themselves.

The term “antibody” refers to immunoglobulin evoked in animals by an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

The terms “homology” and “homologous” as used herein in reference to amino acid sequences refer to the degree of identity of the primary structure between two amino acid sequences. Such a degree of identity may be directed a portion of each amino acid sequence, or to the entire length of the amino acid sequence. Two or more amino acid sequences that are “substantially homologous” may have at least 50% identity, preferably at least 75% identity, more preferably at least 85% identity, most preferably at least 95%, or 100% identity.

The terms “binding component”, “molecule of interest”, “agent of interest”, “ligand” or “receptor” as used herein may be any of a large number of different molecules, biological cells or aggregates, and the terms are used interchangeably. Each binding component may be immobilized on a solid substrate and binds to an analyte being detected. Proteins, polypeptides, peptides, nucleic acids (nucleotides, oligonucleotides and polynucleotides), antibodies, ligands, saccharides, polysaccharides, microorganisms such as bacteria, fungi and viruses, receptors, antibiotics, test compounds (particularly those produced by combinatorial chemistry), plant and animal cells, organdies or fractions of each and other biological entities may each be a binding component.

The term “bind” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring.

### BRIEF DESCRIPTION OF THE FIGURES

FIG. 1 presents an illustrative schematic of one embodiment of a BCRAM molecule. 1—Protein A: 2—Acceptor Peptide; 3—Ovalbumin Fragment; 4—Murine specific α-IgM.

FIG. 2 presents an illustrative schematic of one embodiment of a BCRAM molecule. 1—Protein A: 2—Acceptor Peptide; 3—Ovalbumin Fragment; 4—Human specific α-IgM.

FIG. 3 presents several embodiments of a BCRAM-autoantigen-BCR complex:

FIG. 3A: A murine BCRAM-antibody-autoantigen-BCR complex.

FIG. 3B: A murine BCRAM-RNA antibody-autoantigen-BCR complex.

FIG. 3C: A murine BCRAM-DNA antibody-autoantigen-BCR complex.

FIG. 3A: A human BCRAM-antibody autoantigen-BCR complex.

FIG. 3B: A human BCRAM-RNA antibody-autoantigen-BCR complex.

FIG. 3C: A human BCRAM-DNA antibody-autoantigen-BCR complex.

FIG. 4 presents exemplary data showing proliferation of wild type (WT) murine B cells, but not TLR7/9 KO B cells, with a BCRAM comprising an α-chromatin specific autoantibody.

FIG. 5 shows several prior art compositions that have been reported to stimulate B cell populations by:

FIG. 5A: Small molecule ligands.

FIG. 5B: A mixture of DNA-specific and RNA-specific autoantibodies.

FIG. 5C: α-IgM-coupled DNAs.

FIG. 5D: A mixture of bifunctional α-IgM/DNA-specific and α-IgM/RNA-specific autoantibodies.

### DETAILED DESCRIPTION OF THE INVENTION

The present invention is related to compositions and methods to stimulate the immune system. For example, antigen-specific antibodies may be produced by stimulating B cell populations with specific antigenic compounds, such as an adjuvant comprising a macromolecule capable of activating a Toll-Like receptor (TLR). For example, a BCR adapter IgM (BCRAM) is described to exemplify delivery of autoantigens to polyclonal B cell populations resulting in immunoactivation by TLR activation. Alternatively, a compound is described that inhibits TLR activation.

### I. Immunoregulatory Control Mechanisms

Mammalian immune systems are believed to use pattern recognition receptors (PRR) to detect molecular patterns expressed by infectious microorganisms (e.g., for example, epitopes). For example, when a PRR encounters an epitope the immune system may be stimulated to produce antibodies that have specific binding affinity to that epitope. Consequently, when a biological organism senses the presence of an infectious agent (i.e., for example, a microorganism), the PRRs activate immune cells to proliferate antibodies and mount a defensive response to prevent and/or reduce infection against that particular infectious agent.

A. B Cell Population Activation

B cell responses are initiated by the binding of multivalent antigens to the highly diversified, clonally distributed B cell receptors (BCRs). Antigen-induced clustering of the BCRs triggers a signaling cascade that involves the activation of at least four major signaling pathways that include phospholipase C (PLC), the Rho family of GTPases, ras and phosphatidylinositol-3-kinase (PI3K). Campbell K S., “Signal transduction from the B cell antigen-receptor” Curr Opin Immunol 1999 11:256-264; and Kurosaki T., “Genetic analysis of B cell antigen receptor signaling” Annu Rev Immunol 1999 17:555-559. Combinations of these signaling pathways lead to the activation of MAP kinases and transcription of a variety of genes associated with B cell activation. Dal Porto et al., “B cell antigen receptor signaling 101” Mol Immunol 2004; 41:599-613; and Schulze-Luehnnann et al., “Antigen-receptor signaling to nuclear factor kappa B” Immunity 2006; 25:701-715. In addition, the BCR, under the influence of signaling cascades, efficiently transports antigens to a MHC-class II-containing, multivesicular, intracellular compartment where the antigens are proteolytically cleaved and the resulting peptides are assembled with MHC class II molecules into complexes for recognition by helper T cells. Lanzavecchia A., “Receptor-mediated antigen uptake and its effect on antigen presentation to class II-restricted T lymphocytes” Annu Rev Immunol 1990; 8:773-793.

Recent studies provided evidence that the correct targeting of the BCR to class II-containing intracellular compartments was dependent on the activation of phospholipase D (PLD), a lipase implicated in the intracellular trafficking of a variety of surface receptors. Snyder et al., “A mutation in Epstein-Barr virus LMP2A reveals a role for phospholipase D in B-Cell antigen receptor trafficking” Traffic 2006; 7:993-1006; Melendez et al., “Phospholipase D and immune receptor signalling” Semin Immunol. 2002 February; 14(1):49-55; and Jenkins et al., “Phospholipase D: a lipid centric review” Cell Mol Life Sci 2005; 62:2305-2316. Antigen binding to the BCR also appears to result in the clustering or fusion of intracellular vesicles resulting in large LAMP-1 positive, MHC-class II-rich compartments into which the BCR traffics, although the molecular basis of this subcellular reorganization is not known. Lankar et al., “Dynamics of major histocompatibility complex class II compartments during B cell receptor-mediated cell activation” J Exp Med 2002; 195:461-472; and Siemasko et al., “The control and facilitation of MHC class II antigen processing by the BCR” Curr Opin Immunol 2001; 13:32-36.

Lastly, it was shown recently that in antigen presenting cells MHC-class II-containing intracellular compartments continuously fuse with the autophagosomes, providing means for presentation of cytosolic proteins on MHC-class II molecules. Schmid et al., “Fc receptors and their interaction with complement in autoimmunity” Immunol Lett 2005; 100:56-67. It has becoming increasingly clear that signaling through the BCR is regulated, or fine-tuned by, an array of signaling receptors receiving information from the B cells' environment.

In particular, receptors of the innate immune systems have been shown to significantly influence the outcome of antigen engagement by the BCR and may, in fact, contribute to the immune dysregulation observed in autoimmune diseases. Bernasconi et al., “A role for Toll-like receptors in acquired immunity: up-regulation of TLR9 by BCR triggering in naive B cells and constitutive expression in memory B cells” Blood 2003; 101:4500-4504. Of particular interest are the Toll-like receptors (TLRs), germ line encoded innate immune system receptors that recognize conserved molecular patterns of microorganisms. Akira et al., “Toll-like receptors: critical proteins linking innate and acquired immunity” Nat Immunol 2001; 2:675-680.

Autoreactive B cells are present in the lymphoid tissues of healthy individuals, but typically remain quiescent. When this homeostasis is perturbed, the formation of self-reactive antibodies can have pathological consequences. For example, B cells expressing an antigen receptor specific for a self-immunoglobulin-gamma (IgG) make a class of autoantibodies known as rheumatoid factor (RF). Activation of RF⁺ B cells has been reported to be mediated by IgG2a-chromatin immune complexes and a synergistic engagement of an antigen receptor with a member of the MyD88-dependent Toll-like receptor (TLR) family. Leadbetter et al., “Chromatin-IgG complexes activate B cells by dual engagement of IgM and Toll-like receptors” Nature 2002 416(6881):603-7. Inhibitor studies implicate that the activated Toll-like receptor may be TLR9. Such observations provide a link between an innate and adaptive immune systems in the development of systemic autoimmune disease and may help to explain the preponderance of autoantibodies reactive with nucleic acid-protein particles. Exploitation of this dual-engagement pathway would be expected to develop therapies that specifically target autoreactive B cells.

A role for CpG dinucleotides in autoreactive B cell stimulation was first demonstrated in studies examining synthetic phosphodiester (PD)-linked oligonucleotides (ODNs). Kuramoto et al., 1992 “Oligonucleotide sequences required for natural killer cell activation” Jpn. J. Cancer Res. 83:1128-1131; and Messina et al., 1993 “The influence of DNA structure on the in vitro stimulation of murine lymphocytes by natural and synthetic polynucleotide antigens” Cell. Immunol. 147:148-157. Subsequent studies examining short synthetic phosphorothioate (PS) ODNs led to the identification of PuPuCGPyPy (Pu, purine; Py, pyrimidine) as a useful motif for engagement of mouse TLR9. Krieg et al., 2002. “CpG motifs in bacterial DNA and their immune effects” Annu. Rev. Immunol. 20:709-760.

However, a series of recent studies have questioned how well these PS-stabilized CpG motifs reflect authentic microbial and/or endogenous ligands. For example, when used at exceedingly high concentrations, PD-linked non-CpG ODNs can have stimulatory activity. Vollmer et al., 2002 “Highly immunostimulatory CpG-free oligodeoxynucleotides for activation of human leukocytes” Antisense Nucleic Acid Drug Dev. 12: 165-175; Elias et al., 2003 “Strong cytosine-guanosine-independent immunostimulation in humans and other primates by synthetic oligodeoxynucleotides with PyNTTTTGT motifs” J. Immunol. 171:3697-3704; and Yasuda et al., 2005 “Endosomal translocation of vertebrate DNA activates dendritic cells via TLR9-dependent and -independent pathways” J. Immunol. 174: 6129-6136. Moreover, total mammalian DNA was reported to activate a TLR9 fusion protein expressed on the surface of transfected HEK 293 cells. Barton et al., 2006 “Intracellular localization of Toll-like receptor 9 prevents recognition of self DNA but facilitates access to viral DNA” Nat. Immunol. 7:49-56. Furthermore, total mammalian DNA complexed with the antimicrobial peptide LL37 was found to stimulate plasmacytoid dendritic cells (DCs). Lande et al., 2007 “Plasmacytoid dendritic cells sense self-DNA coupled with antimicrobial peptide” Nature 449:564-569. Additionally, PD-ODNs were recently reported to activate DCs through a sequence-independent, backbone-dependent mechanism. Haas et al., 2008 “The DNA sugar backbone 2′ deoxyribose determines Toll-like receptor 9 activation” Immunity 28:315-323. Nevertheless, the role of mammalian DNA CpG content in the activation of TLR9, and in particular in the activation of autoreactive B cells, remains unresolved; either the relative activities of CpG-rich and non-CpG-rich mammalian DNA have not been accurately compared between experimental systems that depend on the delivery of DNA by the addition of a 3′-poly(G) tail to force aggregation or by artificial delivery to early endosomes with the transfection reagent DOTAP. Yasuda et al., 2005 “Endosomal translocation of vertebrate DNA activates dendritic cells via TLR9-dependent and -independent pathways” J. Immunol. 174: 6129-6136; and Kerkmann et al., 2005 “Spontaneous formation of nucleic acid-based nanoparticles is responsible for high interferon-α induction by CpG-A in plasmacytoid dendritic cells” J. Biol. Chem. 280:8086-8093.

B. Toll-Like Receptors

Detection of molecular patterns by members of the Toll-like receptor (TLR) gene family has been reported to be involved in the activation of the immune response. Medzhitov et al., 1997. “A human homologue of the Drosophila Toll protein signals activation of adaptive immunity” Nature 388:394-397. TLRs can also recognize self-antigens released from stressed or damaged host tissues, and such self-recognition can potentially promote the development of autoimmune disease. As previously reported, immune complexes (ICs) comprising IgG bound to mammalian chromatin have been shown to activate transgenic rheumatoid factor (RF) B cells through a process that may involve B cell antigen receptor (BCR) recognition of the IC and subsequent delivery of the DNA to TLR9 sequestered in an endosomal/lysosomal compartment. Leadbetter et al., 2002. “Chromatin-IgG complexes activate B cells by dual engagement of IgM and Toll-like receptors” Nature 416:603-607; and Marshak-Rothstein 2004 “Comparison of CpG s-ODNs, chromatin immune complexes, and dsDNA fragment immune complexes in the TLR9-dependent activation of rheumatoid factor B cells” J. Endotoxin. Res. 10:247-251.

These same low affinity RF⁺ B cells do not proliferate in response to protein-containing ICs. Similarly, chromatin ICs, but not protein ICs, stimulate myeloid and plasmacytoid DCs to secrete cytokines, in this case through coengagement of FcγRs and TLR9. Boule et al. 2004 “Toll-like receptor 9-dependent and -independent dendritic cell activation by chromatin immunoglobulin G complexes” J. Exp. Med. 199:1631-1640; and Means et al., 2005 “Human lupus autoantibody-DNA complexes activate DCs through cooperation of CD32 and TLR9” J. Clin. Invest. 115:407-417. Moreover, under the appropriate conditions, mammalian chromatin can also directly stimulate DNA-reactive cells, again through a TLR9-dependent process. Viglianti et al. 2003 “Activation of autoreactive B cells by CpG dsDNA. Immunity 19:837-847. Thus, it has been suggested that autoantibodies reactive with DNA or DNA-associated proteins are the earliest and most prevalent serological markers of SLE in both animal models and human disease. Burlingame et al., 1994 “The central role of chromatin in autoimmune responses to histones and DNA in systemic lupus erythematosus” J. Clin. Invest. 94:184-192.

RNA and RNA/protein macromolecules, such as Sm/RNP, constitute a category of autoantigen frequently targeted in systemic autoimmune diseases such as systemic lupus erythematosus (SLE). Sm/RNP particles comprise uridine-rich U1 RNA bound by Sm and other associated proteins. Recent studies have identified TLR7 and TLR8 as receptors for viral single-stranded (ss) RNA, and uridine-rich ssRNA was found to be a ligand. Diebold et al., 2004 “Innate antiviral responses by means of TLR7-mediated recognition of single-stranded RNA” Science 303:1529-1531; and Heil et al., 2004 “Species-specific recognition of single-stranded RNA via Toll-like receptor 7 and 8” Science 303: 1526-1529. These data raised the possibility that a BCR/TLR9 paradigm, previously established for chromatin/DNA activation of autoreactive B cells and DCs, would also apply to TLR7/8 involvement in the activation of B cells by RNA-associated autoantigens.

An interaction between BCR and TLR9 may recognize unmethylated CpG-DNA motifs present in viral and bacterial DNA. As for the BCR, TLR9-initiated signaling ultimately results in the activation of the MAP kinases, p38 and JNK, and NF-κB, although through signaling pathways distinct from those triggered by the BCR. Indeed, CpG DNA-induced TLR9 signaling has been shown to synergize with antigen-induced BCR signaling in the phosphorylation of p38 and JNK, and NF-κB activation. Moreover, this synergistic response was also evident when measured by cell proliferation and Ig secretion. Yi et al., “Convergence of CpG DNA- and BCR-mediated signals at the c-Jun N-terminal kinase and NF-kappaB activation pathways: regulation by mitogen-activated protein kinases” Int Immunol 2003; 15:577-591. The synergistic engagement of TLR9 and the BCR in response to DNA-containing antigens has been implicated in the activation of autoimmune B cells. Leadbetter et al., “Chromatin-IgG complexes activate B cells by dual engagement of IgM and Toll-like receptors” Nature 2002; 416:603-607; and Viglianti et al., “Activation of autoreactive B cells by CpG dsDNA” Immunity 2003; 19:837-847.

Many of the autoantigens targeted in systemic lupus erythematosus (SLE) contain self DNA, histones, RNA or ribonucleoproteins that are thought to be released from apoptotic cells. Moreover, TLR9 has been shown to play a role in regulating DNA-specific autoantibody production in mouse models of lupus by mechanisms that involve the simultaneous engagement of TLR9 and the BCR. Christensen et al., “Toll-like receptor 9 controls anti-DNA autoantibody production in murine lupus” J Exp Med 2005; 202:321-331; and Christensen et al., “Toll-like receptor 7 and TLR9 dictate autoantibody specificity and have opposing inflammatory and regulatory roles in a murine model of lupus” Immunity 2006; 25:417-428. The relevance of TLR9 signaling in B cells to human diseases is underscored by the observation that genetic variations in TLR9 are linked to SLE susceptibility. Tao et al., “Genetic variations of Toll-like receptor 9 predispose to systemic lupus erythematosus in Japanese population” Ann Rheum Dis 2007; 66:905-909. Consequently, blocking TLR9 signaling in autoreactive B cells has been suggested as a therapy for SLE. Lenert, P. S., “Targeting Toll-like receptor signaling in plasmacytoid dendritic cells and autoreactive B cells as a therapy for lupus” Arthritis Res Ther 2006; 8:203.

A role for TLRs in autoantibody response does not appear to be limited to the DNA-binding TLR9, but also includes the endosomal RNA-binding TLR, TLR7. RNA-associated autoantigens activate B cells to produce antibodies in a BCR- and TLR7-dependent manner. Lau et al., RNA-associated autoantigens activate B cells by combined B cell antigen receptor/Toll-like receptor 7 engagement” J Exp Med 2005; 202:1171-117. Recently, genetic evidence for a role of TLR7 in autoimmune diseases was provided in mice by the finding that a duplication of the TLR7 gene resulted in RNA-specific autoantibody response. Pisitkun et al., “Autoreactive B cell responses to RNA-related antigens due to TLR7 gene duplication” Science 2006; 312:1669-16. It was subsequently shown that a simple two fold increase in expression of TLR7 alone was sufficient to accelerate autoimmunity. Deane et al., “Control of Toll-like Receptor 7 Expression Is Essential to Restrict Autoimmunity and Dendritic Cell Proliferation” Immunity 2007; 27:801-810. Thus, understanding the molecular basis of the interaction of BCR and TLRs in response to DNA- and RNA-containing antigens facilitates an understanding of the molecular basis of autoantibody responses.

At present, little is known about the cellular or molecular mechanisms that underlie TLR9- and TLR7-enhanced signaling of the BCR, and given the spatial segregation of the BCR on the plasma membrane and both TLR9 and TLR7 in early endosomal compartments it is not obvious how hyper-responses to DNA- or RNA-containing antigens are achieved. Ahmad-Nejad et al., “Bacterial CpG-DNA and lipopolysaccharides activate Toll-like receptors at distinct cellular compartments” Eur J Immunol 2002; 32:1958-1968.

One family of PPRs (supra) are the toll-like receptors (TLRs), represented by TLR7 and TLR9 which are believed to have evolved to sense RNA and DNA, respectively. In one embodiment, the present invention contemplates an adjuvant comprising a macromolecule capable of activating a TLR. In one embodiment, the TLR activation results in immunostimulation. In one embodiment, the present invention contemplates an composition comprising a compound, wherein the compound inhibits TLR activation.

For example, mouse models of systemic lupus erythematosus (SLE) develop antibodies to DNA, RNA and associated proteins. It is believed that the antibodies are produced through mechanisms that depend on B cell receptor (BCR) binding of an autoantigen-associated macromolecule. It is further believed that a BCR-autoantigen complex translocates to an endolysosomal compartment comprising TLRs (i.e., for example, TLR7 and/or TLR9). Although it is not necessary to understand the mechanism of an invention, it is believed that a BCR-autoantigen complex stimulates the TLR via autoantigen binding as opposed to a direct BCR-TLR interaction. As a result, the functional outcome of this form of stimulation may result from “crosstalk” between the BCR and TLR7 and/or TLR9 signaling cascades, and differs from BCR/TLR7 vs BCR/TLR9 activation.

Inactivation of TLR9 and TLR7 genes within the endosomal/lysosomal compartments of autoimmune prone mice results in the reduction of autoantibody specificity for DNA/RNA and associated proteins such that the mice have less severe disease. In addition to other autoimmune diseases, the detection of antibodies against DNA and RNA are used clinically to diagnose systemic lupus erythematosus (SLE).

Currently, those in the art have suggested four (4) basic strategies to model the pathogenic processes leading to autoantibody production in vitro:
(1) Small ligands—stimulation of polyclonal B cells with low molecular weight ligands, e.g., CpG ODN 1826 for TLR9, or R848 (and derivatives) for TLR7, that are endocytosed via poorly characterized means and specifically stimulate TLR9 or TLR7;
(2) Autoantibodies—stimulation of IgG2a-reactive B cells, e.g., rheumatoid factor⁺ from AM14 transgenic mice, with IgG2a autoantibodies of the a allotype (Lau et al. “RNA-associated autoantigens activate B cells by combined B cell antigen receptor/Toll-like receptor 7 engagement” J Exp Med 202:1171-1177 (2005); and Leadbetter et al., “Chromatin-IgG complexes activate B cells by dual engagement of IgM and Toll-like receptors” Nature 416:603-607 (2002);
(3) Anti-BCR/DNA conjugates—stimulation of polyclonal B cells with conjugates of polyclonal or monoclonal anti-BCR antibodies with stimulatory ODN or dsDNA fragment (Uccellini et al., “Autoreactive B cells discriminate CpG-rich and CpG-poor DNA and this response is modulated by IFN-alpha” J Immunol 181:5875-5884 (2008); Chaturvedi et al., “The B cell receptor governs the subcellular location of Toll-like receptor 9 leading to hyperresponses to DNA-containing antigens” Immunity 28:799-809 (2008): and Green et al., “Activation of autoreactive B cells by endogenous TLR7 and TLR3 RNA ligands” J Biol Chem 287:39789-39799 (2012); and
(4) Bifunctional antibodies—stimulation of polyclonal B cells with bifunctional antibodies in which one variable domain is derived from the H and L chain of an autoantibody and the other variable domain is derived from the H and L chains of a monoclonal antibody reactive with IgM (the BCR) (REF—MTA with Abbvie).

### II. B Cell Receptor/Toll Like Receptor Immunostimulatory Platforms

Activation of autoreactive B cells involves an internalization of ligands (i.e., for example, autoantigens such as nucleic acids) and delivery of these ligands to the Toll-like Receptor (TLR) contained in the endolysosomal compartment. For example, ribonucleoproteins represent a large fraction of autoantigens in systemic autoimmune diseases. Many uridine-rich mammalian RNA sequences associated with common autoantigens can effectively activate autoreactive B cells. Furthermore, priming B cells with type I interferon (IFN) may increase the magnitude of activation, and the range over which RNAs are stimulatory. RNAs that contain a high degree of self-complementarity can also activate B cells through TLR3. On the other hand, RNA sequences that activate predominantly through TLR7, the activation is proportional to uridine-content, and more precisely defined by the frequency of specific uridine-containing motifs. These results suggest the existence of parameters that define specific mammalian RNAs as different ligands for TLRs.

Synergistic engagement of the B cell receptor (BCR) and Toll-like receptor 9 (TLR9) in response to DNA-containing antigens underlies the production of many autoantibodies in systemic autoimmune diseases. However, the molecular basis of this synergistic engagement is not known. Given that these receptors are spatially segregated, with a BCR on the cell surface and a TLR9 in endocytic vesicles, any mechanism to achieve this synergy is likely complex. It has been reported that upon antigen binding, the BCR initiates signaling at the plasma membrane and continues to signal to activate MAP kinases as the BCR-antigen complex internalizes and traffics to autophagosome-like compartments. The internalized BCR-antigen complex also signals through a phospholipase-D-dependent pathway to recruit TLR9-containing endosomes to the autophagosome via the microtubular network. Chaturvedi et al., “The B cell receptor governs the subcellular location of Toll-like receptor 9 leading to hyperresponses to DNA-containing antigens” Immunity 2008 28(6):799-809. Although it is not necessary to understand the mechanism of an invention it is believed that the recruitment of TLR9 to the autophagosomes plays a role in the hyperactivation of MAP kinases, such that BCR-induced TLR9 recruitment resulting in B cells hyper-responses may provide new targets for therapeutics for autoimmune diseases.

Presently, a clear understanding of the actual pathways involved in autoreactive B cell activation is not known in the art. Consequently, it has been difficult to determine the most appropriate therapeutic strategy. In some embodiments, the present invention was developed on a step-wise strategic outline, as follows:
(1) identify the elements of the relevant pathways;
(2) evaluate murine models in which the BCR and/or TLR signaling components have been deleted or amplified by gene targeting;
(3) evaluate the specificity of human tumors driven by gain of function mutations in TLR signaling components;
(4) evaluate in vitro the therapeutic efficacy of drugs designed to interfere with BCR/TLR-dependent activation of tumors and/or autoreactive B cells;
(5) monitor the loss of pathogenic autoantibodies in patients being treated for autoimmune diseases.

In some embodiments, the present invention contemplates compositions and methods to stimulate B cell populations using the above therapeutic strategy that have advantages over the previously reported concepts. See, Table 1.

TABLE 1
Comparison of Prior Art Strategies to BCRAM

| | | | anti-IgM/ | | |
|---|---|---|---|---|---|
| | Small | | DNA | Bifunctional | |
| | ligands | Autoantibodies | conjugates | antibodies | BCRAM |
| B cell targets | Polyclonal | AM14 BCR | Polyclonal | Polyclonal | Polyclonal |
| | | Tg | | | |
| Mimics BCR/TLR | No | Yes | Partly | Yes | Yes |
| coengagement of | | | | | |
| autoreactive B cells by an | | | | | |
| autoantigen | | | | | |
| Application to both TLR9 | Yes | Yes | No | Yes | Yes |
| and TLR7 engaging | | | (Unstable) | | |
| ligands | | | | | |
| Only works with mouse | NA | Yes | No | No | No |
| IgG2a^{a/j} | | | | | |
| Works on any B6 gene- | Yes | No | Yes | Yes | Yes |
| targeted mouse needs to | | (BALB/c) | | | |
| be backcrossed | | | | | |
| Adaptable to both mouse | NA | No | Not | Yes | Yes |
| and human IgG | | (Mouse) | Tested | | |
| antibodies | | | | | |
| Depends on biotinylation | NA | No | Yes | No | No |
| of mAb and batch to | | | | | |
| batch variation | | | | | |
| Requires cloning of each | NA | No | No | Yes | No |
| autoantibody H and L | | | | | |
| chain and | | | | | |
| construction/optimization | | | | | |
| of each individual | | | | | |
| bifunctional antibody | | | | | |
| Can be used to screen | NA | No | No | No | Yes |
| numerous existing human | | | | | |
| and mouse monoclonal | | | | | |
| autoAbs and also serum | | | | | |
| samples - monitor | | | | | |
| response to therapy | | | | | |
| Can be used to screen | NA | No | No | No | Yes |
| receptor antibodies of B | | | | | |
| cell tumors resulting from | | | | | |
| gain-of-function | | | | | |
| mutations in TLR | | | | | |
| signaling components | | | | | |

In one embodiment, the present invention contemplates a method comprising stimulating a plurality of polyclonal B cell populations with an autoantigen capable of binding to a BCR, TLR7 and/or TLR9. In one embodiment, the polyclonal B cell population is a human polyclonal B cell population. In one embodiment, the polyclonal B cell population is a murine polyclonal B cell population. In one embodiment, the autoantigen recapitulates autoreactive antibody production.

In one embodiment, the present invention contemplates a composition capable of delivering an autoantibody to a B cell. In one embodiment, the composition comprises a BCR adapter IgM (BCRAM). Although it is not necessary to understand the mechanism of an invention, it is believed that a BCRAM can deliver any autoantibody, from nearly any source, to any B cell population (i.e., for example, human and/or murine B cell population).

A. The BCR Adapter IgM Complex

In one embodiment, BCRAM has the ability to bind to a BCR. In one embodiment, the BCRAM has the ability to bind to other IgG antibodies. For example, BCRAM may bind to IgG antibodies derived from autoimmune mice or human patients exhibiting at least one symptom of an autoimmune disease.

In one embodiment, a BCRAM comprises an IgG variable domain. In one embodiment, the IgG variable domain comprising an H chain, an L chain or an H chain and an L chain. In one embodiment, the IgG variable domain is specific for an IgM antibody. In one embodiment, the IgM antibody is a B cell receptor.

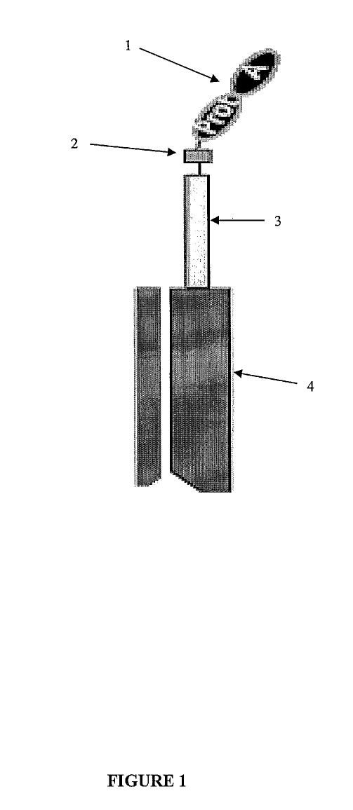

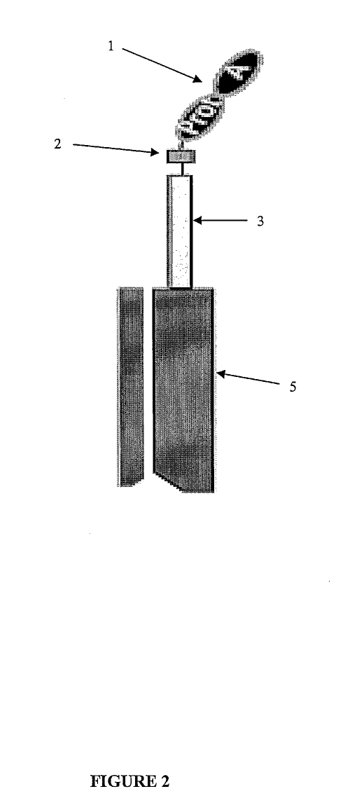

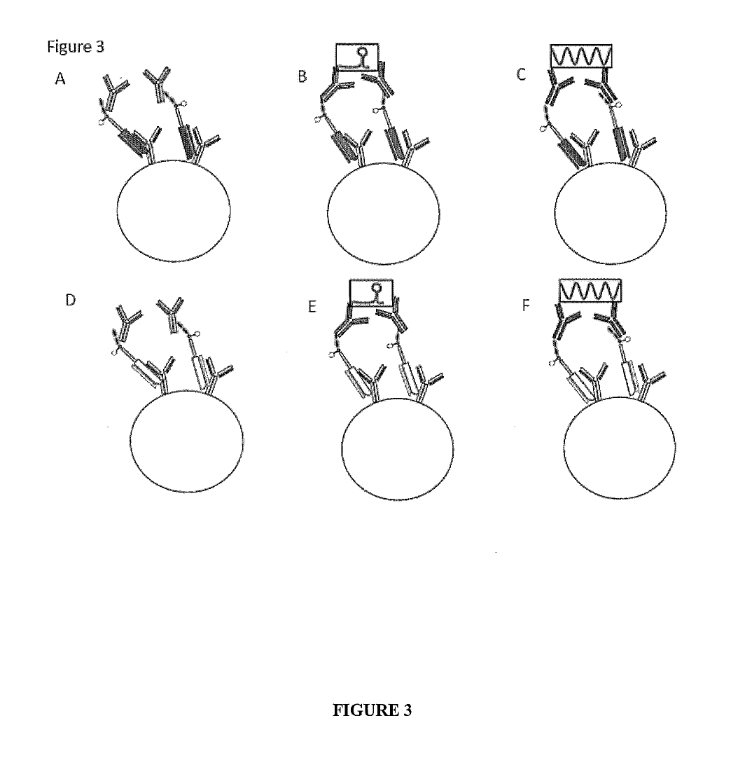

In one embodiment, a BCRAM comprises an IgG Fc-binding domain. In one embodiment the IgG Fe-binding domain comprises Protein A or Protein G. See, FIG. 1 and FIG. 2. In one embodiment, the IgG Fc binding domain binds to an autoantibody. In one embodiment, the autoantibody is an RNA-specific autoantibody. In one embodiment, the autoantibody is an RNA-associated autoantibody. In one embodiment, the autoantibody is a DNA-specific autoantibody. In one embodiment, the autoantibody is a DNA-associated autoantibody. In one embodiment, the autoantibody is a murine autoantibody. In one embodiment, the autoantibody is a human autoantibody. See, FIGS. 3A-3F. In one embodiment, the autoantibody is derived from a murine exhibiting at least one symptom of an autoimmune disease. In one embodiment, the autoantibody is derived from a human exhibiting at least one symptom of an autoimmune disease. In one embodiment, the autoantibody binds to an autoantigen. In one embodiment, the autoantigen is a type 1 diabetes autoantigen. In one embodiment, the type 1 diabetes autoantigen includes, but is not limited to, PDX1, AnT8, CHGA IAAP, GAD(65) and/or DiaPep277. In one embodiment, the autoantigen is an alopecia areata autoantigen. In one embodiment, the alopecia areata autoantigen includes, but is not limited to, keratin 16, K18585, M10510, J01523, 022528, D04547, 005529, B20572 and/or F11552. In one embodiment, the autoantigen is a systemic lupus erythematosus autoantigen. In one embodiment, the systemic lupus erythematosus autoantigen includes, but is not limited to, TRIM21/Ro52/SS-A1 and/or histone H2B. In one embodiment, the autoantigen is a Behçet's disease autoantigen. In one embodiment, the Behçet's disease autoantigen includes, but is not limited to, S-antigen, alpha-enolase, selenium binding partner and/or Sip1 C-ter. In one embodiment, the autoantigen is a Sjögren's syndrome autoantigen. In one embodiment, the Sjögren's syndrome autoantigen includes, but is not limited to, La/SSB, KLK11 and/or a 45-kd nucleus protein. In one embodiment, the autoantigen is a rheumatoid arthritis autoantigen. In one embodiment, the rheumatoid arthritis autoantigen includes, but is not limited to, vimentin, gelsolin, alpha 2 HS glycoprotein (AHSG), glial fibrillary acidic protein (GFAP), α1B-glycoprotein (A1BG), RA33 and/or citrullinated 31F4G1. In one embodiment, the autoantigen is a Grave's disease autoantigen. In one embodiment, the autoantigen is an antiphospholipid antibody syndrome autoantigen. In one embodiment, the antiphospholipid antibody syndrome autoantigen includes, but is not limited to, zwitterionic phospholipids, phosphatidyl-ethanolamine, phospholipid-binding plasma protein, phospholipid-protein complexes, anionic phospholipids, cardiolipin, β2-glycoprotein I (β2GPI), phosphatidylserine, lyso(bis)phosphatidic acid, phosphatidylethanolamine, vimentin and/or annexin A5. In one embodiment, the autoantigen is a multiple sclerosis autoantigen. In one embodiment, the multiple sclerosis autoantigen includes, but is not limited to, myelin-associated oligodendrocytic basic protein (MOBP), myelin basic protein (MBP), myelin proteolipid protein (PLP), myelin oligodendrocyte glycoprotein (MOG) and/or alpha-B-crytallin. In one embodiment, the autoantigen is an irritable bowel disease autoantigen. In one embodiment, the irritable bowel disease autoantigen includes, but is not limited to, a ribonucleoprotein complex, a small nuclear ribonuclear polypeptide A and/or Ro-5,200 kDa. In one embodiment, the autoantigen is a Crohn's disease autoantigen. In one embodiment, the Crohn's disease autoantigen includes, but is not limited to, zymogen granule membrane glycoprotein 2 (GP2), an 84 bp allele of CTLA-4 (AT)ₙ repeat polymorphism, MRP8, MRP14 and/or complex MRP8/14. In one embodiment, the autoantigen is a dermatomyositis autoantigen. In one embodiment, the dermatomyositis autoantigen includes, but is not limited to, aminoacyl-tRNA synthetases, Mi-2 helicase/deacetylase protein complex, signal recognition particle (SRP), T2F1-γ, MDA5, NXP2, SAE and/or HMGCR. In one embodiment, the autoantigen is an ulcerative colitis autoantigen. In one embodiment, the ulcerative colitis autoantigen includes, but is not limited to, 7E12H12 and/or M(r) 40 kD autoantigen. In one embodiment, the autoantigen is a synthetic autoantigen. In one embodiment, the autoantigen is a derivatized autoantigen. In one embodiment, the autoantigen is a conjugated autoantigen.

In one embodiment, the BCRAM comprises an ovalbumin fragment. Although it is not necessary to understand the mechanism of an invention the ovalbumin component enables a comparison of BCR/TLR9 versus BCR/TLR7 crosslinking. Such comparisons allow examination of antigen processing and presentation pathways.

In one embodiment, a BCRAM comprises an acceptor site. In one embodiment, the acceptor site is a BIR acceptor site. In one embodiment, the acceptor site is biotinylated. It is believed that a biotinylated acceptor site can bind to a fluorochrome-conjugated strepaviden and thereby monitor uptake and intracellular trafficking of a BCRAM complex.

Preliminary data has shown that BCRAM binds to a murine BCR via a murine autoantibody-antigen complex. Additionally, a construct has been designed for a BCRAM for binding to a human BCR via a human autoantibody-antigen complex. Demonstration of this respective binding ability means that: (a) both mouse and human autoantibodies (e.g., extracted from serum), or the serum alone, could be used to stimulate wild-type (WT) and gene-targeted mice (i.e., for example, knock-out (KO) mice), and (b) both mouse and human autoantibodies (e.g., extracted from serum), or the serum alone, could be used to compare normal healthy control individuals to immunodeficient, autoimmune, or at risk patients.

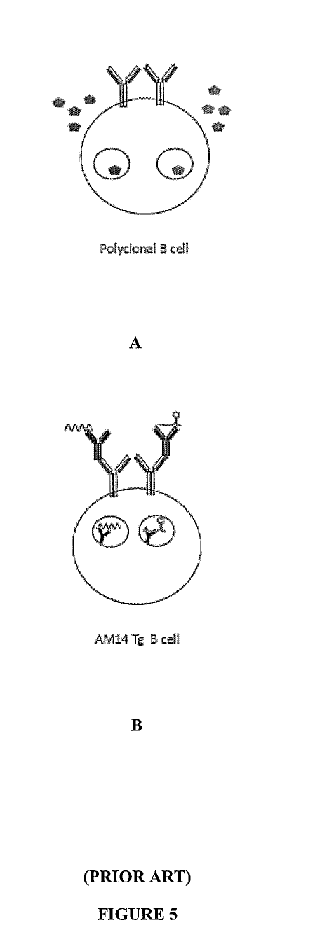

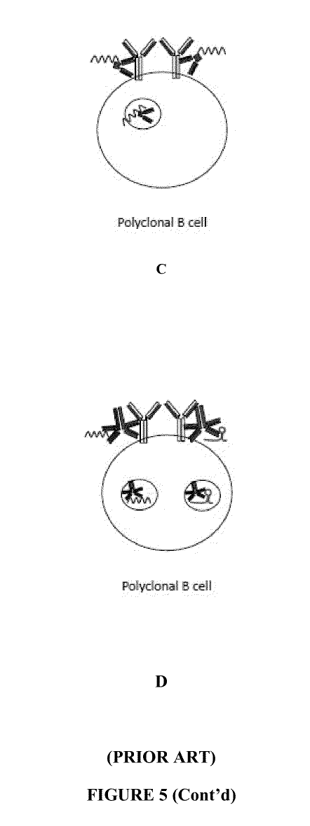

One advantage of some of the presently contemplated embodiments over currently known immunostimulation methods is an ability to specifically stimulate B cells through BCR engagement combined with clinically relevant TLR7 and/or TLR9 ligands. For example, to model B cell activation in patients with SLE, healthy control- or patient-isolated cells can be treated with a specific BCRAM mixed with patient serum. In comparison, conventional compositions that have been reported to stimulate B cell populations are: i) small molecule ligands (See, FIG. 5A); ii) a mixture of DNA-specific and RNA-specific autoantibodies (e.g., the AM14 BCR Tg B cell system; see, FIG. 5B); iii) α-IgM-coupled DNA (See, FIG. 5C); and iv) a mixture of bifunctional α-IgM/DNA-specific and α-IgM/RNA-specific autoantibodies (See, FIG. 5D).

Each of the above cited conventional compositions for stimulating B cell populations have specific disadvantages, for example: (1) small ligands do not engage the BCR and therefore do not mimic the physiologically relevant signaling cascades; (2) AM14 BCR Tg B cell systems are limited to murine IgG2a autoantibodies of the appropriate allotypes and cannot be used to test any other gene-targeted phenotypes, for example, those on a B6 background; (3) anti-BCR/DNA conjugates only bind to TLR9, and anti-BCR/RNA conjugates are unstable; and (4) bifunctional antibodies could, in theory, be utilized for studying multiple RNA associated autoantibodies, however, the methods for construction are complex because for each antibody the H and L chains need to be cloned and inserted into the appropriate backbone.

The presently disclosed BCRAM technology has advantages over all the above conventional compositions due to an overall B cell target flexibility and the IgG Fc binding domain allows binding to a wide variety of autoantibodies enabling population-wide BCR binding and/or stimulation. The general applicability of BCRAM allows a use for comparing and contrasting interspecies autoantibodies and B cells (e.g., for example, mice versus humans). Further the BCRAM technology allows comparing and contrasting of serum samples from patients with various autoimmune diseases (i.e., for example, before, during and after therapy). In some embodiments, the BCRAM complex further comprises biotin to facilitate drug delivery to an endolysosomal compartment.

### III. Autoimmune Diseases

Autoimmune diseases arise from an inappropriate immune response of the body against substances and tissues normally present in the body (e.g., autoimmunity). This may be restricted to certain organs (e.g. in autoimmune thyroiditis) or involve a particular tissue in different places. The treatment of autoimmune diseases is typically with immunosuppression—medication that decreases the immune response. A large number of autoimmune diseases are recognized of which a representative number are discussed in detail below.

A. Type 1 Diabetes

Type 1 diabetes is a prototypic, organ-specific autoimmune disease. Diverse antigen-specific immunotherapy using insulin or glutamic acid decarboxylase peptides and other immunotherapies, such as antibodies, fusion proteins, cytokines, regulatory T cells, small-molecule inhibitors, nonspecific immune modulators, or dietary modifications, have been attempted in human type 1 diabetes. Some of these immunotherapies delay the onset of diabetes or reduce insulin requirements or blood glucose level in patients with established type 1 diabetes. However, most of these immunotherapies failed to induce complete remission of established type 1 diabetes, which could be due to: 1) technical difficulties in the achievement of immune tolerance to diabetic autoantigens or in the inhibition of autoimmune responses to those antigens that can be applied to human patients without significant adverse effects; and 2) markedly reduced β-cell mass at the time of disease onset that should be replenished. Kim et al., “Immunotherapeutic treatment of autoimmune diabetes” Crit Rev Immunol. 2013; 33(3):245-81.

Type 1 diabetes mellitus (T1DM) is characterized by recognition of beta cell proteins as self-antigens, called autoantigens (AAgs), by patients' own CD4+ and CD8+ T cells and/or the products of self-reactive B cells, called autoantibodies. These AAgs are divided into two categories on the basis of beta-cell-specificity. The list of the targets associated with beta cell-specific AAgs is continuously growing. Many T1DM-associated AAgs are well characterized and have important clinical applications for disease prediction, diagnosis, and antigen-specific tolerance immunotherapy. Identification of T1DM-associated AAgs provides insight into the pathogenesis of T1DM and to understanding the clinical aspects of the disease. Recently discovered T1DM-Aags include, but are not limited to PDX1, ZnT8, CHGA, and IAAP. Han et al., “Novel autoantigens in type 1 diabetes” Am J Transl Res. 2013 5(4):379-392.

Although no cure for diabetes presently exists, the onset of insulitis can be diminished in the non-obese diabetic (NOD) mouse type 1 diabetes model by inoculation with endogenous β-cell autoantigens. These include the single peptide vaccines insulin, GAD(65) (glutamic acid decarboxylase), and DiaPep277 (an immunogenic peptide from the 60-kDa heat shock protein). DiaPep277 is the only autoantigen so far to demonstrate positive results in human clinical trials. Diamyd (an alum adjuvant+recombinant GAD(65) protein formulation) has shown great promise for suppressing β-cell autoreactivity in phase I and II clinical trials. Nicholas et al., “Autoantigen based vaccines for type 1 diabetes” Discov Med. 2011 11(59):293-301. Insulin is also suggested to be an autoantigen for the development of Type 1 diabetes. Nakayama et al., “Insulin as a key autoantigen in the development of type 1 diabetes” Diabetes Metab Res Rev. 2011 27(8):773-777.

Type 1 diabetes can occur at any age. However, it is most often diagnosed in children, adolescents, or young adults. Insulin is a hormone produced by special cells, called beta cells, in the pancreas. The pancreas is found behind your stomach. Insulin is needed to move blood sugar (glucose) into cells, where it is stored and later used for energy. In type 1 diabetes, beta cells produce little or no insulin. Without enough insulin, glucose builds up in the bloodstream instead of going into the cells. The body is unable to use this glucose for energy. This leads to the symptoms of type 1 diabetes that include but are not limited to, thirst, hunger, fatigue, blurred eyesight, loss of feeling or tingling in the feet, unexplained weight loss and/or frequent urination.

B. Alopecia Areata

Alopecia areata (AA) is an autoimmune disease resulting in the premature arrest of the follicular growth cycle clinically resulting in patchy, non-scarring hair loss. The presence of a dense follicular T cell infiltrate and variations in cytokines have led to the hypothesis that T cell activation and alterations in inflammatory mediators participate in the etiopathogenesis of the disease. AA pathogenesis is believed to have a dominant TH1-mediated component, with potential involvement of the TH17 pathway. However, a fully integrated view of intersecting cytokine networks that support the autoimmune response in AA is lacking A more precise understanding of cytokine pathways in disease is required to rationally explore cytokine targeted treatment strategies. Giordano et al., “Cytokine pathways and interactions in alopecia areata” Eur J Dermatol. 2013 Jun. 24. [Epub ahead of print]. AA-reactive HF-specific antigens were isolated from normal human scalp anagen HF extracts by immunoprecipitation using serum antibodies from 10 AA patients. Samples were analyzed by LC-MALDI-TOF/TOF mass spectrometry, which indicated strong reactivity to the hair growth phase-specific structural protein trichohyalin in all AA sera. Keratin 16 (K16) was also identified as another potential AA-relevant target HF antigen. Leung et al., “Trichohyalin is a potential major autoantigen in human alopecia areata” J Proteome Res. 2010 9(10):5153-5163. In one study, 10 single sera were screened against the human protein filter array containing 37,200 human proteins, and 48 IgG-specific and 32 IgG3-specific AA autoantigens were identified. The quantitative validation using highly sensitive protein microarrays leads to the confirmation of 10 IgG- and 6 IgG3-specific autoantigens. For example, putative alopecia areata specific autoantigens include, but are not limited to, K18585 (Accession No. AV652428; cDNA clone GLCDACO5), M10510 (Accession No. XM031401 EGF-like domain), J01523 (Accession No. AK022755 cDNA FLJ12693 fis clone NT2RP1000324), 022528 (Accession. No. NM004436 Endosulfine (ENSA)), K24594 (Accession No. NM003134 Signal recognition particle subunit), D04547 (Accession No. NM022965 FGFR3), 005529 (Accession No. M91670 Keratinocyte ubiquitin carrier protein (E2-EPF)), M17541 (Accession No. BC006318 Erythrocyte membrane protein band), B20572 (Accession No. NM007029 Neuron-specific growth-associated protein (SCG10)), or F11552 (Accession No. NM006769 LM04). Lueking et al., “Profiling of alopecia areata autoantigens based on protein microarray technology” Mol Cell Proteomics 2005 4(9):1382-1390.

The cause of alopecia areata is unknown but can occur in men, women and children. About 1 in 5 people with this condition have a family history of alopecia. In a few people, hair loss may occur after a major life event such as an illness, pregnancy, or trauma. Forms of alopecia include, but are not limited to, alopecia areata—patches of hair loss; alopecia totalis—complete loss of scalp hair, and/or alopecia universalis—total loss of all body hair.

Hair loss is usually the only symptom of alopecia, but a burning sensation or itching may also be present Alopecia areata usually begins as one to two patches of hair loss. Hair loss, is most often seen on the scalp. It may also occur in the beard, eyebrows, and arms or legs in some people. Patches where hair has fallen out are smooth and round in shape. They may be peach-colored. Hairs that look like exclamation points are sometimes seen at the edges of a bald patch.

C. Systemic Lupus Erythematosus

Systemic lupus erythematosus (SLE) is a prototypic autoimmune disease, characterized by the production of autoantibodies against multiple organs. MicroRNAs (miRNAs) are non-coding, single-stranded small RNAs that post-transcriptionally regulate gene expression. Evidence is accumulating that miRNAs play a role in the pathogenesis of SLE. miRNAs may also play regulatory roles in the DNA methylation pathway, type I interferon pathway, estrogen and regulatory T-cells in the pathogenesis of SLE. Ma et al., “MicroRNAs in the pathogenesis of systemic lupus erythematosus” Int J Rheum Dis. 2013 16(2):115-121.

TRIM21/Ro52/SS-A1, a 52-kDa protein, is an autoantigen recognized by antibodies in sera of patients with SLE and Sjögren's syndrome (SS), another systemic autoimmune disease, and anti-TRIM21 antibodies have been used as a diagnostic marker for decades. TRIM21 belongs to the tripartite motif-containing (TRIM) super family, which has been found to play important roles in innate and acquired immunity. Yoshimi et al., “Autoantigen TRIM21/Ro52 as a Possible Target for Treatment of Systemic Lupus” Int J Rheumatol. 2012:718237. A major epitope has been identified in the known TRIM21 SLE autoantibody that spans the central

TRIM21 region having the amino acid sequence LQ-ELEKDEREQLRILGE-KE. Protein structure investigations, secondary structure predictions, and surface area calculations revealed that the best matching partial sequence to fulfill all primary and secondary structure requirements was the four amino acid spanning motif ‘L-E-Q-L’, which is present in both the sequential and the α-helical peptide conformation. Al-Majdoub et al., “Mass spectrometric and peptide chip characterization of an assembled epitope: analysis of a polyclonal antibody model serum directed against the Sjøgren/systemic lupus erythematosus autoantigen TRIM21” J Mass Spectrom. 2013 48(6):651-659.

Histone H2B is a common target of autoantibodies in both spontaneous and drug-induced systemic lupus erythematosus (SLE). Asp(25) of histone H2B (H2B) may spontaneously convert to an isoaspartic acid (isoAsp) in vivo. Analysis of serum from lupus-prone mice and histone antibody positive SLE patients revealed antibodies specific to the Asp and isoAsp H2B(21-35) peptide, and that the expression of these antibodies is dependent on TLR9. IsoAsp H2B(21-35) is immunogenic in non-autoimmune prone mice and mice lacking the ability to repair isoAsp have significantly reduced levels of antibodies to H2B. Doyle et al., “Autoimmunity to isomerized histone H2B in systemic lupus erythematosus” Autoimmunity 2013 46(1):6-13.

SLE is a long-term autoimmune disorder that may affect the skin, joints, kidneys, brain, and other organs. SLE is much more common in women than men. It may occur at any age, but appears most often in people between the ages of 10 and 50. African Americans and Asians are affected more often than people from other races.

Symptoms vary from person to person, and may come and go. Almost everyone with SLE has joint pain and swelling. Some develop arthritis. Frequently affected joints are the fingers, hands, wrists, and knees. Common SLE symptoms include, but are not limited to, chest pain when taking a deep breath, fatigue, unexplained fever, general discomfort, uneasiness, malaise, hair loss, mouth sores, sunlight sensitivity, skin rash (e.g., a “butterfly” rash over the cheeks and bridge of the nose), or swollen lymph nodes.

D. Behçet's Disease

Behçet's disease (BD) is a multisystem inflammatory disorder of uncertain origin, although it remains defined within the spectrum of systemic immune-mediated vasculitic disorders and also represents a spectrum of putative autoimmune disease. Major symptoms include oral aphthous ulcers, genital ulcerations, skin lesions, and ocular lesions. Despite afflicting many systems, ocular complications of BD are some of the more devastating for the patient and their quality of life. Eye involvement, which affects 60-80% of BD patients, is characterized in its more severe form by posterior or panuveitis including occlusive retinal vasculitis. While pathogenesis of BD remains complex, association with Class I MHC (HLA-B*51) predisposing to inflammation with engagement of the innate-immune system (neutrophils, NK cells), and perpetuated by the adaptive T cell responses against infectious- and/or auto-antigens. Despite the choice of conventional immunosuppressive therapies available, only recently with the advent of biologic therapy has visual prognosis and outcomes been substantially and favorably altered. For example, both interferon-α (IFN-α) and tumour necrosis factor (TNF)-α antagonists deliver promising results and for the first time improve prognosis. With IFN-α therapy, durable remissions of uveitis can be achieved and lead to drug-free remission. Similarly, anti-TNF therapy with infliximab is reported to be rapidly effective in inducing and maintaining remission. Most recently, rising evidence reports on the use of adalimumab, etanercept, and golimumab, while use of anti-interleukin (IL)-1 agents (anakinra, canakinumab, gevokizumab), IL-6 blockers (tocilizumab), and rituximab (depleting anti-CD20 antibody) is also increasing. Mesquida et al., “Current and future treatments for Behçet's uveitis: road to remission” Int Ophthalmol. 2013 Jun. 1. [Epub ahead of print].

Retinal autoantigens were recognized by sera from BD patients with uveitis using a proteomic technique, 2-dimensional electrophoresis (2DE) followed by Western blotting (WB). Six protein spots showing high reactivity with the serum from the BD patients were detected as candidate retinal autoantigens, and three of them were identified by mass spectrometry; S-antigen, alpha-enolase and selenium binding protein (SBP). Okunuki et al., “Proteomic surveillance of autoimmunity in Behçet's disease with uveitis: selenium binding protein is a novel autoantigen in Behçet's disease” Exp Eye Res. 2007 84(5):823-831. A cDNA library from human microvascular endothelial cells was screened with serum IgG from two patients with Behçet's disease that isolated a reactive clone specific to the carboxy-terminal subunit of Sip1 (Sip1 C-ter). Using ELISA, IgG, IgM and IgA specific to Sip1 C-ter was measured in patients with various autoimmune diseases characterized by the presence of serum anti-endothelial cell antibodies, such as Behçet's disease, systemic lupus erythematosus, systemic sclerosis and various forms of primary vasculitis, as well as in patients with diseases that share clinical features with Behçet's disease, such as inflammatory bowel disease and uveitis. Delunardo et al., “Identification and characterization of the carboxy-terminal region of Sip-1, a novel autoantigen in Behçet's disease” Arthritis Res Ther. 2006; 8(3):R71. Serum from a BD patient was used as a probe to immunoscreen a lambdaZAP expression cDNA library. Candidate autoantigen cDNAs were characterized by direct nucleotide sequencing and their expressed products were examined for reactivity to the entire panel of BD sera using immunoprecipitation. Reactivity was also examined with normal control sera and disease control sera from patients with lupus and Sjögren's syndrome. Six independent candidate clones were isolated from the cDNA library screen and were identified as overlapping partial human kinectin cDNAs. The finding that kinectin was an autoantigen was verified in 9 out of 39 (23%) BD patient sera. Lu et al., “Identification of kinectin as a novel Behçet's disease autoantigen” Arthritis Res Ther. 2005; 7(5):R1133-1139.

E. Sjögren's Syndrome

Sjogren syndrome is an autoimmune disorder in which the glands that produce tears and saliva are destroyed, causing dry mouth and dry eyes. However, the condition may affect many different parts of the body, including the kidneys and lungs.

Activated IFN-1 pathway plays a part in the autoimmune disease process of Sjögren's syndrome; therefore, several therapies aiming to reduce or inhibit the IFN-1 production and its effects may be a target for future treatment plans. Activated aryl hydrocarbon receptor may interact with latent Epstein-Barr virus (EBV) infection, which in turn may predispose to the development of Sjögren's syndrome. It is estimated that the population is 95% positive for EBV serology. Microbial factors may incite autoimmune disease. Although this hypothesis is proven in a few illnesses such as rheumatic fever, there is no definitive evidence of an infectious environmental trigger in Sjögren's syndrome. However, there are circumstantial data with regard to viruses and several potential mechanisms of disease. These include antigen mimicry, polyclonal lymphocyte activation, and infection-mediated innate end-organ inflammation. In addition, hepatitis C virus infection clearly causes a Sjögren's-syndrome-like illness. Igoe et al., “Autoimmunity and infection in Sjögren's syndrome” Curr Opin Rheumatol. 2013 July; 25(4):480-7.

Sjögren syndrome (SS) antigen B (SSB)/La was identified as a pre-miRNA-binding protein that regulates miRNA processing in vitro. All three RNA-binding motifs (LAM, RRM1, and RRM2) of La/SSB are required for efficient pre-miRNA binding. Intriguingly, La/SSB recognizes the characteristic stem-loop structure of pre-miRNAs, of which the majority lack a 3′ UUU terminus. Liang et al., “Sjogren syndrome antigen B (SSB)/La promotes global microRNA expression by binding microRNA precursors through stem-loop recognition” J Biol Chem. 2013 288(1):723-736. Sera from 11 patients diagnosed with SS, 8 patients with dry eye disease (DED), and 8 normal age/sex-matched controls (NL) were collected for detecting antibodies against various tissue kallikreins; KLK1, KLK11, KLK12, and KLK13 by capture enzyme-linked immunosorbent assay. As antibodies to KLK11 were elevated in SS patients, this suggests that KLK11 is a putative autoantigen. El Annan et al., “Elevated immunoglobulin to tissue KLK11 in patients with Sjögren syndrome” Cornea 2013 32(5):e90-93. One study reports that the 120-kd alpha-fodrin autoantigen is also present in a shorter fragment, indicating that there may be a distinct apoptosis-related protease that cleaves alpha-fodrin in the lacrimal gland. A novel salivary gland-specific autoantibody was detected in 50.9% of sera from SS patients. Another autoantigen may comprise a 45-kd nucleus protein.

Symptoms may include, but are not limited to, dryness of the mouth and eyes, itching eyes, feeling that something is in the eye, difficulty swallowing or eating, loss of sense of taste, difficulty speaking, thick or stringy saliva, mouth sores or pain, hoarseness, fatigue, fever, Color change of hands or feet, joint pain or joint swelling and/or swollen glands.

F. Rheumatoid Arthritis

Rheumatoid arthritis (RA) is a chronic, inflammatory, autoimmune disease with typical onset between the ages of 40 and 50 years, characterized by chronic inflammation in synovial joints. Effective treatment for RA is lacking because the clear etiology and pathogenesis of RA have not been fully elucidated. Cytokine-mediated immunity has been found to play an important role in the pathogenesis of various autoimmune diseases such as RA. Recently, IL-32 is identified with high expression in RA patients and mice models of experimental inflammatory arthritis. IL-32 is recognized to play a crucial role in RA with pro-inflammatory effects. Furthermore, interventions for blocking IL-32 in RA seem possible and applicable. Therefore, targeting IL-32 may give therapeutic potential. Xu et al., “IL-32 with potential insights into rheumatoid arthritis” Clin Immunol. 2013 147(2):89-94.

Increasing levels of physical activity (PA) have been shown to decrease inflammation, reduce pain, increase functional ability and improve self-esteem in people with RA. Health behaviour change (HBC) interventions have recently shown promise in facilitating the promotion of PA within a range of long-term conditions. There is currently no evidence synthesis relating to HBC interventions to increase PA in the RA population. Cramp et al., “Health Behaviour Change Interventions for the Promotion of Physical Activity in Rheumatoid Arthritis: A Systematic Review” Musculoskeletal Care 2013 May 7 [Epub ahead of print].

An immune-proteomic strategy was implemented to identify putative RA autoantigens. Synovial fluid samples from clinically diagnosed RA patients were separated on two-dimensional gel electrophoresis (2-DE). Samples from patients with non-RA rheumatisms (osteoarthritis and trauma) were used as controls. Immunoreactive proteins were spotted by Western blotting followed by identification through Q-TOF mass spectrometer analysis. Forty Western blots were generated using plasma from ten individual RA patients and 33 reactive spots were identified, 20 from the high molecular weight (HMW) gel and 13 from the low molecular weight (LMW) gel. Among the 33 common immunogenic spots, 18 distinct autoantigens were identified, out of which 14 are novel proteins in this context. Expression analysis of five proteins, vimentin, gelsolin, alpha 2 HS glycoprotein (AHSG), glial fibrillary acidic protein (GFAP), and α1B-glycoprotein (A1BG) by Western blot analysis using their specific antibodies revealed their higher expression in RA synovial fluid as compared to non-RA samples. Further analysis revealed that GFAP and A1BG can be proposed as potential new autoantigens of diagnostic importance for RA subjects. Biswas et al., “Identification of novel autoantigen in the synovial fluid of rheumatoid arthritis patients using an immunoproteomics approach” PLoS One 2013; 8(2):e56246. The activation of CD4(+) T cells specific for the RA autoantigen, proteoglycan aggrecan, following antigen presentation by DC was compared between macrophages and B cells. Aggrecan-specific B cells are equally efficient APC as DC and macrophages and use similar intracellular antigen-processing pathways. Antigen presentation by aggrecan-specific B cells to TCR transgenic CD4(+) T cells also results in enhanced CD4(+) T cell interferon-γ production and Th1 effector sub-set differentiation compared with that seen with DC. Wilson et al., “Presentation of the candidate rheumatoid arthritis autoantigen aggrecan by antigen-specific B cells induces enhanced CD4(+) T helper type 1 subset differentiation” Immunology 2012 135(4):344-354. The protein termed RA33 was determined to be one major autoantigen in rheumatoid arthritis (RA) patients and antiRA33 auto-antibodies were found to appear shortly after onset of RA. The monoclonal antiRA33 antibody is located in the aa79-84 region on recombinant RA33; the epitope sequence is MAARPHSIDGRVVEP. Sequence comparisons of the 15 best scoring peptides from the peptide chip analysis revealed that the epitope can be separated into two adjacent binding parts. The N-terminal binding parts comprise the amino acid residues “DGR”, resembling the general physico-chemical properties “acidic/polar-small-basic”. The C-terminal binding parts contain the amino acid residues “VVE”, with the motif “hydrophobic-gap-acidic”. El-Kased et al., “Mass spectrometric and peptide chip epitope mapping of rheumatoid arthritis autoantigen RA33” Eur J Mass Spectrom (Chichester, Eng). 2009; 15(6):747-759. The human chondrocyte cDNA expression library was citrullinated by PADI4 and was immunoscreened with anti-modified citrulline antibodies and sera from patients with rheumatoid arthritis. One immunoreactive cDNA clone containing a 2480-base pair insert was isolated and sequence analysis revealed that the cDNA included a part of the eukaryotic translation initiation factor 4G1. Immunoreactivity against a recombinant citrullinated eIF4G1 fragment was observed with high specificity in 50.0% of RA patients. The levels of antibodies against citrullinated eIF4G1 were correlated with those of anti-CCP antibodies. Citrullinated eIF4G1 was identified as a candidate citrullinated autoantigen in RA patients. Citrullination of eIF4G1 may thus be involved in the pathogenesis of RA. Okazaki et al., “Identification of citrullinated eukaryotic translation initiation factor 4G1 as novel autoantigen in rheumatoid arthritis” Biochem Biophys Res Commun. 2006 3; 341(1):94-100.

RA usually affects joints on both sides of the body equally. Wrists, fingers, knees, feet, and ankles are the most commonly affected. The disease often begins slowly, usually with only minor joint pain, stiffness, and fatigue.

Joint symptoms of RA may include, but are not limited to, morning stiffness, which lasts more than 1 hour, unused joints feel warm, tender, and stiff, loss of joint range of motion, joint deformation, pleurisy, dry eyes and mouth, eye burning, itching, and discharge, skin nodules, numbness, tingling, or burning in the hands and feet, and/or sleep difficulties.

G. Graves' Disease

Graves' disease is the most common cause of hyperthyroidism. It is caused by an abnormal immune system response that causes the thyroid gland to produce too much thyroid hormone. Graves' disease is most common in women over age 20. However, the disorder may occur at any age and may affect men as well. The thyroid gland is an important organ of the endocrine system. It is located in the front of the neck just below the voice box. This gland releases the hormones thyroxine (T4) and triiodothyronine (T3), which control body metabolism. Controlling metabolism is critical for regulating mood, weight, and mental and physical energy levels. If the body makes too much thyroid hormone, the condition is called hyperthyroidism whereas an underactive thyroid leads to hypothyroidism.

Graves' disease (GD) is a systemic autoimmune disease that is characterized by hyperthyroidism, orbitopathy and in rare cases dermopathy. Graves' orbitopathy (GO) is an inflammatory disease of eye and orbit which occurs in about 30-60% of patients. Hyperthyroidism occurs due to the presence of stimulating TSHR-autoantibodies (TRAbs) leading to increased serum levels of thyroid hormones. Attempts to induce Graves' disease in mice by immunization against the hTSHR or its variants have resulted in production of TRAbs that stimulate thyroid follicular cells to increase thyroid hormone secretion. Graves' like orbital changes, such as inflammation, adipogenesis and muscle fibrosis are more difficult to induce. Wiesweg et al., “Current Insights into Animal Models of Graves' Disease and Orbitopathy” Horm Metab Res. 2013 Apr. 23. [Epub ahead of print].

Symptoms of Grave's disease include, but are not limited to, anxiety, male breast enlargement, difficulty concentrating, double vision, exophthalmos, eye irritation and tearing, fatigue, frequent bowel movements, goiter, heat intolerance, increased appetite, increased sweating, insomnia, irregular menstrual periods, muscle weakness, nervousness, palpitations, arrhythmia, restlessness, difficulty sleeping, shortness of breath with activity, tremor and/or unexplained weight loss.

H. Antiphospholipid Antibody Syndrome

Antiphospholipid antibody syndrome (APS) is an autoimmune disease that leads to arterial and/or venous thrombosis, recurrent pregnancy loss and persistently positive aPLs. Antibodies against antigenic anionic phospholipid protein complexes are detected by their reactivity to the anionic phospholipids (or protein phospholipid complexes) in solid-phase immunoassays or by their property of inhibiting phospholipid-dependent coagulation reactions (the “lupus anticoagulant” effect).

Patients with clinical manifestations highly suggestive of APS but persistently negative conventional aPLs are classified as having seronegative APS. Ongoing research has revealed the existence of non-criteria antibodies proposed to be relevant to APS and that can be potentially included in the disease's classification criteria. Some antibodies of this heterogeneous aPL family may include, but are not limited to, antibodies to a zwitterionic phospholipid, namely phosphatidylethanolamine, phospholipid-binding plasma proteins, phospholipid-protein complexes and anionic phospholipids other than cardiolipin. Although these molecules can increase the diagnostic yield of APS, their clinical relevance is still debatable and needs to be confirmed by interlaboratory efforts toward standardizing diagnostic tools, in addition to experimental data and larger longitudinal studies. Nayfe et al., “Seronegative antiphospholipid syndrome” Rheumatology (Oxford). 2013 Mar. 15. [Epub ahead of print].

Enzyme-linked immunosorbent assays for anticardiolipin and anti-β2-glycoprotein I antibodies and clotting assays for the lupus anticoagulant are the tests recommended for detecting aPL. However, the aPL are a heterogeneous group of antibodies directed against anionic phospholipids but also toward phospholipid-binding plasma proteins or phospholipid-protein complexes. β2-glycoprotein I (β2GPI) is an antigen of APS, however during apoptosis, lysophospholipids can become exposed on the cell surface, and mainly through their interaction with β2GPI, they can become targets of aPL. Some CL metabolites are likely to escape from the remodeling cycle. This may account for the progressive loss of mitochondrial CL during apoptosis, as well as for the presence of CL and lyso-CL at the cell surface, where they can interact with β2GPI and become targets of aPL. Other recognized targets of aPL are represented by phosphatidylserine, lyso(bis)phosphatidic acid, phosphatidylethanolamine, vimentin, and annexin A5. These molecules may allow improving the knowledge on the pathogenesis, and the early identification of APS. Although several studies have shown the presence of antibodies directed against other antigens in APS, their clinical relevance is still a matter of debate, and it needs to be confirmed with experimental data and longitudinal studies. Alessandri et al., “New autoantigens in the antiphospholipid syndrome” Autoimmun Rev. 2011 August; 10(10):609-616.

I. Multiple Sclerosis

Multiple sclerosis (MS) affects women more than men. The disorder is most commonly diagnosed between ages 20 and 40, but can be seen at any age. MS is caused by damage to the myelin sheath, the protective covering that surrounds nerve cells. When this nerve covering is damaged, nerve signals slow down or stop. The nerve damage is caused by inflammation. Inflammation occurs when the body's own immune cells attack the nervous system. This can occur along any area of the brain, optic nerve, and spinal cord.

Multiple sclerosis (MS) is assumed to be an autoimmune disease initiated by autoreactive T cells that recognize central nervous system antigens. Although adaptive immunity is clearly involved in MS pathogenesis, innate immunity increasingly appears to be implicated in the disease. Natural killer (NK) cells may be involved in immunoregulation in MS, leading to the question of whether a particular NK cell subtype will account for this effect. Changes of NK cell functionality in MS can be associated with MS activity, and depletion of NK cells exacerbated the course of disease in a murine model of MS, experimental autoimmune encephalomyelitis. A deficiency and transient “valleys” in NK cell killing activity in human MS, may coincide with symptomatic relapse. However, the molecular basis of the defect in killing activity has not been determined. For example, perforin is expressed in CD16(+) NK cells while there is an inverse relationship between myelin loaded phagocytes and the proportion of CD 16(+) NK cells expressing perforin in the circulation. This inverse relationship is consistent with a role for NK cell killing activity in dampening autoimmunity. On the other hand, it has been broadly reported that first line MS therapies, such as interferon-beta, glatiramer acetate as well as escalation therapies such as fingolimod, daclizumab, or mitoxantrone seem to affect NK cell functionality and phenotype in vivo. Chanvillard et al., “The role of natural killer cells in multiple sclerosis and their therapeutic implications” Front Immunol. 2013; 4:63.

Myelin-associated oligodendrocytic basic protein (MOBP) is as an important candidate autoantigen in MS as the others including, but not limited to, myelin basic protein (MBP), myelin proteolipid protein (PLP), and myelin oligodendrocyte glycoprotein (MOG). Kaushansky et al., “The myelin-associated oligodendrocytic basic protein (MOBP) as a relevant primary target autoantigen in multiple sclerosis” Autoimmun Rev. 2010 9(4):233-236. Serum IgG, IgA and IgM repertoires were compared against a range of eye lens-derived ocular antigens using sera from healthy control subjects and MS patients with or without uveitis. This comparison revealed that among ocular antigens, alpha B-crystallin is the dominant target antigen for serum autoantibodies in both MS patients and healthy controls and might represent an autoantigen for MS. van Noort et al., “Autoantibodies against alpha B-crystallin, a candidate autoantigen in multiple sclerosis, are part of a normal human immune repertoire” Mult Scler. 2006 12(3):287-293.

Symptoms vary, because the location and severity of each attack can be different. Episodes can last for days, weeks, or months. These episodes alternate with periods of reduced or no symptoms (remissions). Fever, hot baths, sun exposure, and stress can trigger or worsen attacks. It is common for the disease to return (relapse). However, the disease may continue to get worse without periods of remission. Because nerves in any part of the brain or spinal cord may be damaged, patients with multiple sclerosis can have symptoms in many parts of the body. Symptoms of MS include, but are not limited to, loss of balance, muscle spasms, numbness or abnormal sensation in any area, problems moving arms or legs, problems walking, problems with coordination and making small movements, tremor in one or more arms or legs, weakness in one or more arms or legs, constipation, stool leakage, difficulty beginning to urinate, frequent need to urinate, strong urge to urinate, incontinence, double vision, eye discomfort, uncontrollable rapid eye movements, vision loss, facial pain, painful muscle spasms, tingling, crawling, or burning feeling in the arms and legs, decreased attention span, poor judgment, and memory loss, difficulty reasoning and solving problems, depression or feelings of sadness, dizziness and balance problems, hearing loss, slurred or difficult-to-understand speech, trouble chewing and swallowing and/or fatigue.

J. Irritable Bowel Disease

Functional gastrointestinal disorders are those in which no abnormal metabolic or physical processes, which can account for the symptoms, can be identified. The irritable bowel syndrome (IBS) is a significant functional disorder, which affects 10-20 percent of the population worldwide. Predominant symptoms of IBS are abnormal defecation associated with abdominal pain, both of which may be exacerbated by psychogenic stress.

In one study, eighty-seven percent (87%) of IBS sera and fifty-nine (59%) of control sera contained anti-enteric neuronal antibodies. Antibody immunostaining was seen in the nucleus and cytoplasm of neurons in the enteric nervous system. Protein microarray analysis detected antibody reactivity for autoantigens in serum with anti-enteric neuronal antibodies and no reactivity for the same autoantigens in samples not containing anti-enteric neuronal antibodies. Antibodies in sera from IBS patients recognized only 3 antigens out of an 8,000 immunoprotein array. The 3 antigens were: (1) a nondescript ribonucleoprotein (RNP-complex); (2) small nuclear ribonuclear polypeptide A; and (3) Ro-5,200 kDa. These results suggest that symptoms in a subset of IBS patients might be a reflection of enteric neuronal damage or loss, caused by circulating anti-enteric autoimmune antibodies. Wood et al., “Anti-enteric neuronal antibodies and the irritable bowel syndrome” J Neurogastroenterol Motil. 2012 18(1):78-85.

Irritable bowel syndrome (IBS) is a highly prevalent disorder that is characterized by chronic abdominal pain and altered bowel habit. The diagnosis of IBS has traditionally been made by matching the complaints of the patient with established clinical criteria, since the underlying pathophysiology was not known. While the florid inflammation characteristic of inflammatory bowel disease is absent in IBS, changes suggesting immune activation are present in nearly all IBS patients. It has been suggested that IBS is an autoimmune disease such that there is a state of immune activation triggered by intestinal bacterial overgrowth. Al-Khatib et al., “Immune activation and gut microbes in irritable bowel syndrome” Gut Liver 2009 3(1):14-19.

Irritable bowel syndrome (IBS) is a disorder that leads to abdominal pain and cramping, changes in bowel movements, and other symptoms. IBS is not the same as inflammatory bowel disease (IBD), which includes Crohn's disease and ulcerative colitis. In IBS, the structure of the bowel is not abnormal.

K. Crohn's Disease

Crohn's disease is a form of inflammatory bowel disease (IBD). It usually affects the intestines, but may occur anywhere from the mouth to the end of the rectum (anus). The exact cause of Crohn's disease is unknown but is believed to be an autoimmune disorder. People with Crohn's disease have ongoing (chronic) inflammation of the gastrointestinal tract (GI tract). Crohn's disease may involve the small intestine, the large intestine, the rectum, or the mouth. The inflammation causes the intestinal wall to become thick.

Crohn's disease is often considered an autoimmune condition, based on the observations of a histopathological inflammatory process in the absence of identifiable causal microorganism(s) and that immune-modulating therapeutics result in diminished host-directed inflammatory pathology. However, the evidence for a self-targeted immune response is unproven; thus, the instigating and perpetuating forces that drive this chronic inflammation remain unknown. In recent years, a convergence of findings from different fields of investigation has led to a new paradigm, where Crohn's disease appears to be the consequence of an intrinsic innate immune deficiency. While genomic/postgenomic studies and functional immunologic investigations offer a common perspective, critical details of the processes involved require further elaboration. Vinh et al., “Crohn's as an immune deficiency: from apparent paradox to evolving paradigm” Expert Rev Clin Immunol. 2013 9(1):17-30.

Crohn's disease is a complex disease in which genome, microbiome, and environment interact to produce the immunological background of the disease. Disease in childhood is more extensive and characterized by a rapid progression, leading to severe repercussions in the course of the disorder. Several genetic variations have been associated with an increased risk of developing the disease and most of these are also implicated in other autoimmune disorders. The gut has many tiers of defense against incursion by luminal microbes, including the epithelial barrier and the innate and adaptive immune responses. Moreover, recent evidence shows that bacterial and viral infections, as well as inflammasome genes and genes involved in the autophagy process, are implicated in Crohn's disease pathogenesis. Marcuzzi et al., “Genetic and functional profiling of Crohn's disease: autophagy mechanism and susceptibility to infectious diseases” Biomed Res Int. 2013; 2013:297501.

The major zymogen granule membrane glycoprotein 2 (GP2) was identified as the autoantigen of PABs in Crohn's disease. PAB-positive sera from patients with Crohn's disease (n=42) displayed significantly higher IgG reactivity to rat GP2 in ELISA than either PAB-negative sera (n=31), or sera from patients with ulcerative colitis (n=49), or sera from blood donors (n=69) (p<0.0001, respectively). Twenty-eight (66%) and 18 (43%) of 42 PAB-positive sera demonstrated IgG and IgA reactivity to human recombinant GP2 in IIF, respectively. Patients with PAB-negative Crohn's disease (n=31) were not reactive. GP2 mRNA transcription was significantly higher in colon biopsies from patients with Crohn's disease (n=4) compared to patients with ulcerative colitis (n=4) (p=0.0286). Immunochemical staining confirmed GP2 expression in human colon biopsies from patients with Crohn's disease. Roggenbuck et al., “Identification of GP2, the major zymogen granule membrane glycoprotein, as the autoantigen of pancreatic antibodies in Crohn's disease” Gut 2009 58(12):1620-1628. An 84 bp allele of CTLA-4 (AT)n repeat polymorphism was associated with CD in central China. sCTLA-4 levels were highly expressed in CD, especially in active disease, and were correlated with CRP levels and disease behavior in CD patients. Chen et al., “Association of cytotoxic T lymphocyte associated antigen-4 gene (rs60872763) polymorphism with Crohn's disease and high levels of serum sCTLA-4 in Crohn's disease” J Gastroenterol Hepatol. 2011 26(5):924-930. Monospecific antibodies against the calcium-binding proteins MRP8 and MRP14 and their heterodimer MRP8/14 (epitope 27E10) were used to investigate immunohistochemically the distribution of these proteins in routinely processed small and large bowel tissues from patients with Crohn's disease. MRP8, MRP14, and complex MRP8/14 were demonstrated in most granulocytes and macrophages in active Crohn's disease. Schmid et al., “Immunohistochemical demonstration of the calcium-binding proteins MRP8 and MRP14 and their heterodimer (27E10 antigen) in Crohn's disease” Hum Pathol. 1995 26(3):334-337.

Symptoms depend on what part of the gastrointestinal tract is affected. Symptoms range from mild to severe, and can come and go with periods of flare-ups. The main symptoms of Crohn's disease include, but are not limited to, crampy abdominal pain, fever, fatigue, loss of appetite, pain with passing stool (tenesmus), persistent, watery diarrhea, unexplained weight loss, constipation, eye inflammation, rectal fistulas, joint pain and swelling, mouth ulcers, rectal bleeding and bloody stools, skin lumps or sores (ulcers) and/or swollen gums.

L. Ulcerative Colitis

Ulcerative colitis is a type of inflammatory bowel disease (IBD) that affects the lining of the large intestine (colon) and rectum. The cause of ulcerative colitis is unknown but is believed to be an autoimmune disease. Najafi et al., “Autoimmunity in inflammatory bowel disease: a case of ulcerative colitis with diabetes mellitus, autoimmune hepatitis and autoimmune hypothyroidism” Turk J Pediatr. 2012 54(6):651-653. Ulcerative colitis may affect any age group, although there are peaks at ages 15-30 and then again at ages 50-70. The disease can begin the rectal area, and may involve the entire large intestine over time. It may also start in the rectum and other parts of the large intestine at the same time.

Studies have established reactivity of ulcerative colitis colon-bound IgG antibody with an autoantigen recognized by a novel monoclonal antibody, 7E12H12 (IgM isotype). Purified 7E12H12-IgM was used to raise anti-idiotype antibody in the rabbit, and the presence of disease-specific idiotype(s) was evaluated in patients with ulcerative colitis. F(ab′)2 fragments were prepared from serum-IgG of the rabbit immunized with 7E12H12-IgM. Shah et al., “Development of ulcerative colitis-associated anti-idiotype antibody using a novel monoclonal antibody against a colonic autoantigen” Cell Immunol. 1995 166(1):154-157. An M(r) 40 kD antigen was found in goblet cells of normal terminal ileum and proximal colon but not in rectal goblet cells. By contrast, colonic enterocytes expressed this antigen apically with increasing intensity in a distal direction, expanding to intense cytoplasmic expression in rectal enterocytes. The antigen was also expressed by the epithelium of the fallopian tubes, major bile ducts, gall bladder, and epidermis but not by proximal gastrointestinal tract epithelium or 13 other extra-gastrointestinal organs. Halstensen et al., “Epithelial deposits of immunoglobulin G1 and activated complement colocalise with the M(r) 40 kD putative autoantigen in ulcerative colitis” Gut 1993 May; 34(5):650-657; and Takahashi et al., “Isolation and characterization of a colonic autoantigen specifically recognized by colon tissue-bound immunoglobulin G from idiopathic ulcerative colitis” J Clin Invest. 1985 76(1):311-318.

Ulcerative colitis symptoms vary in severity and may start slowly or suddenly. About half of people only have mild symptoms. Others have more severe attacks that occur more often. Many factors can lead to attacks, including respiratory infections or physical stress. Usual symptoms may include, but are not limited to, abdominal pain and cramping, abdominal sounds (a gurgling or splashing sound heard over the intestine), blood and pus in the stools, diarrhea, fever, tenesmus (rectal pain), unexplained weight loss, gastrointestinal bleeding, joint pain and swelling, mouth sores (ulcers), nausea and vomiting and/or skin lumps or ulcers.

M. Dermatomyositis

Dermatomyositis is a muscle disease characterized by inflammation and a skin rash. It is a type of inflammatory myopathy. Currently, the cause of dermatomyositis is unknown but it is generally believed that the condition may be due to a viral infection of the muscles or an autoimmune disease. Anyone can develop dermatomyositis, but it most commonly occurs in children age 5-15 and adults age 40-60. Women develop this condition more often than men do.

Dermatomyositis is presently considered an inflammatory myopathy as part a group of acquired diseases, characterized by immunoflogistic processes primarily involving the skeletal muscle. According to recent classification criteria, four major diseases have been identified: polymyositis (PM), dermatomyositis (DM), sporadic inclusion body myositis (IBM), and necrotizing autoimmune myositis (NAM). Autoantibodies can be found in the sera of most patients with myositis. Myositis-specific autoantibodies (MSAs) are markers of very specific disease entities within the spectrum of myositis, and target proteins involved in key processes of protein synthesis. Myositis autoantigens comprise the well-defined aminoacyl-tRNA synthetases, the Mi-2 helicase/histone deacetylase protein complex, and the signal recognition particle (SRP) ribonucleoprotein, together with novel targets such as TIF1-γ, MDA5, NXP2, SAE, and HMGCR. Recent studies suggest that autoantigens drive a B cell antigen-specific immune response in muscles. Interestingly, an increased expression of Jo-1 and Mi-2 in regenerating fibers in muscle biopsies from PM and DM patients compared to normal was demonstrated. Myositis autoantigen up-regulation was observed in neoplastic tissues, thus representing a potential link between cancer and autoimmunity in myositis. Non-immunological mechanisms seem to participate to the pathogenesis of inflammatory myopathies; induction of endoplasmic reticulum stress response in response to abnormal muscle regeneration and inflammation has recently been reported in patients with myositis. Ghirardello et al., “Autoantibodies in polymyositis and dermatomyositis” Curr Rheumatol Rep. 2013 15(6):335.

Symptoms of dermatomyositis may include, but are not limited to, difficulty swallowing, muscle weakness, muscle stiffness, muscle soreness, purple/violent upper eyelids, purple/red skin rash and/or shortness of breath. The muscle weakness may appear suddenly or develop slowly over weeks or months. Difficulty may be encountered when the arms are raised over the head, rising from a sitting position and/or climbing stairs. The skin rash may appear over the face, knuckles, neck, shoulders, upper chest, and back.

IV. Antibodies

The present invention uses antibodies (i.e., for example, polyclonal or monoclonal) as targeting ligands for a BCRAM complex. In one embodiment, the present invention provides monoclonal antibodies that specifically bind to a B cell receptor protein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ) may be used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a tumor antigen or autoantibody of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO2 gas. The antibody titer of the supernatant of a hybridoma culture can be measured according to the same manner as described above with respect to the antibody titer of the anti-protein in the antiserum. Separation and purification of a monoclonal antibody can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten. In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times. The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a protein expressed resulting from a virus infection (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

V. Pharmaceutical Compositions

The present invention further provides pharmaceutical compositions comprising a BCRAM complex as contemplated herein. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### EXPERIMENTAL

### Example 1

### BCRAM Stimulation of B Cell Proliferation

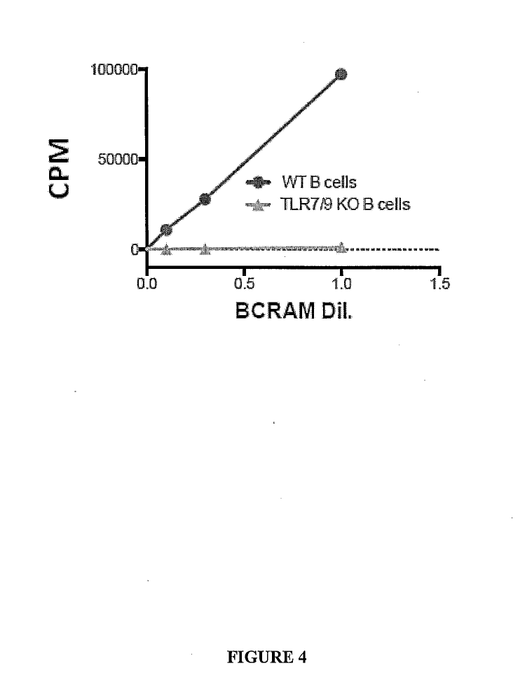

To demonstrate the functionality of BCRAM, TLR-sufficient or TLR7/9 DKO B cells were treated with a combination of (a) increasing concentrations of BCRAM and (b) a constant amount of a monoclonal anti-chromatin antibody. See, FIG. 3C. After 24 hrs, proliferation was assessed by the incorporation of H³-thymidine into replicating DNA. In the absence of BCRAM or TLR7/9 the B cells did not proliferate in response to the anti-chromatin antibody. See, FIG. 4.

### Example II

### B Cell Activation

This example provides one embodiment of a method that can activate a B cell population.

B cells are positively selected from spleen cell suspensions using anti-B220 microbeads (Miltenyi Biotec) and cultured. Leadbetter et al., 2002 “Chromatin-IgG complexes activate B cells by dual engagement of IgM and Toll-like receptors” Nature 416: 603-607. DNA is made single-stranded for addition to 3H9R/Vκ8R B cells by incubation at 95° C. for 10 min, followed by dilution into ice-cold media. F(ab′)₂ goat anti-mouse IgM (Jackson ImmunoResearch Laboratories) is used at 15 μg/ml, PS-ODN 1826 is used at 1 μg/ml, LPS (Sigma-Aldrich) is used at 10 μg/ml, and CLO97 (InvivoGen) is used at 100 ng/ml. Krieg et al., 1998 “Sequence motifs in adenoviral DNA block immune activation by stimulatory CpG motifs” Proc. Natl. Acad. Sci. USA 95:12631-12636. Protein ICs are composed of 5 μg/ml 1D4 and 300 ng/ml biotinylated OVA. In some experiments, cells are primed with IFN-α (PBL InterferonSource, 1000 U/ml) before addition of stimuli. Proliferation is measured with a 6-h pulse of [³H]thymidine 24 h poststimulation. The level of [³H]thymidine incorporation induced by PS-ODN 1826 may range from ˜190,000 to 330,000 cpm, depending on the experiment. Due to this variability, proliferation data averaged from multiple experiments are reported as the percentage proliferation induced by PS-ODN 1826.

We claim:

1. A B Cell Receptor Adapter Immunoglobulin M (BCRAM) complex comprising an IgG variable domain linked to an ovalbumin fragment, wherein said ovalbumin fragment is linked to an acceptor peptide, and wherein said acceptor peptide is linked to an IgG Fc-binding domain.

2. The complex of claim 1, wherein said IgG variable domain has specific affinity for an α-IgM antibody.

3. The complex of claim 2, wherein said α-IgM antibody is a B cell receptor.

4. The complex of claim 1, wherein said IgG variable domain has specific affinity for a murine α-IgM antibody.

5. The complex of claim 4, wherein said murine α-IgM antibody is a murine B cell receptor.

6. The complex of claim 1, wherein said IgG variable domain has specific affinity for a human α-IgM antibody.

7. The complex of claim 6, wherein said human α-IgM antibody is a human B cell receptor.

8. The complex of claim 1, wherein said acceptor site comprises a biotin molecule.

9. The complex of claim 1, wherein said IgG Fc binding domain is linked to an autoantibody.

10. The complex of claim 1, wherein said IgG Fc binding domain is selected from at least one of the group consisting of Protein A and Protein G.

11. The complex of claim 9, wherein said autoantibody binds an autoantigen.

12. The complex of claim 11, wherein said autoantigen is selected from at least one of the group consisting of a ribonucleic acid fragment, a deoxyribonucleic acid fragment, and an α-chromatin fragment.

13. The complex of claim 11, wherein said autoantigen is selected from at least one of the group consisting of, a synthetic autoantigen, a derivatized autoantigen and a conjugated autoantigen.

14. The complex of claim 11, wherein said autoantigen is an autoimmune disease autoantigen.

15. The complex of claim 14, wherein said autoimmune disease autoantigen is selected from at least one of the group consisting of a type 1 diabetes autoantigen, an alopecia areata autoantigen, a systemic lupus erythematosus autoantigen, a Behçet's disease autoantigen, a Sjögren's syndrome autoantigen, a rheumatoid arthritis autoantigen, a Grave's disease autoantigen, an antiphospholipid antibody syndrome autoantigen, a multiple sclerosis autoantigen, an irritable bowel disease autoantigen, a Crohn's disease autoantigen, an ulcerative colitis autoantigen and a dermatomyositis autoantigen.

16. A method for producing autoantigen-specific antibodies comprising;
a) providing;
i) a B Cell Receptor Adaptor Immunoglobulin M (BCRAM) complex,
ii) an autoantibody-autoantigen complex, and
iii) a cell culture wherein at least one cell comprises a B cell receptor (BCR) and a Toll-Like receptor (TLR);
b) binding said autoantibody-autoantigen complex to said BCRAM to form a BCRAM-autoantibody-autoantigen complex;
c) targeting said BCRAM-autoantibody-autoantigen complex to said BCR to form an internalized BCRAM-autoantibody-autoantigen/BCR complex within said at least one cell; and
d) activating said TLR with the internalized BCRAM-autoantibody-autoantigen/BCR complex, wherein autoantigen-specific antibodies are generated.

17. The method of claim 16, wherein said TLR is TLR7.

18. The method of claim 16, wherein said TLR is selected from at least one of the group consisting of TLR9, TLR8 and TLR3.

19. The method of claim 16, wherein said TLR is located in an intracellular compartment of said cell.

20. The method of claim 16, wherein said autoantigen is selected from at least one of the group consisting of a synthetic autoantigen, a derivatized autoantigen and a conjugated autoantigen.

21. The method of claim 16, wherein said autoantigen is an autoimmune disease autoantigen.

22. The method of claim 21, wherein said autoimmune disease autoantigen is selected from at least one of the group consisting of a type 1 diabetes autoantigen, an alopecia areata autoantigen, a systemic lupus erythematosus autoantigen, a Behçet's disease autoantigen, a Sjögren's syndrome autoantigen, a rheumatoid arthritis autoantigen, a Grave's disease autoantigen, an antiphospholipid antibody syndrome autoantigen, a multiple sclerosis autoantigen, an irritable bowel disease autoantigen, a Crohn's disease autoantigen, an ulcerative colitis autoantigen and a dermatomyositis autoantigen.

23. A method for detecting autoimmune disease autoantibodies comprising:
a) providing;
i) a B Cell Receptor Adaptor Immunoglobulin M (BCRAM) complex comprising an autoantibody-autoantigen complex, wherein said autoantibody-autoantigen complex has specific affinity for an autoimmune disease antibody; and
ii) a biological sample derived from a patient, wherein said sample is suspected of comprising said autoimmune disease antibody;
b) contacting said BCRAM complex with said biological sample under conditions such that said autoimmune disease antibody is detected.

24. The method of claim 23, wherein said detection of the autoimmune disease antibody diagnoses an autoimmune disease.

25. The method of claim 24, wherein said autoimmune disease antibody is detected before treatment for said autoimmune disease begins.

26. The method of claim 24, wherein said autoimmune disease antibody is detected after treatment for said autoimmune disease begins.

27. The method of claim 23, wherein said autoantigen is selected from at least one of the group consisting of a synthetic autoantigen, a derivatized autoantigen and a conjugated autoantigen.

28. The method of claim 23, wherein said autoantigen is an autoimmune disease autoantigen.

29. The method of claim 28, wherein said autoimmune disease autoantigen is selected from at least one of the group consisting of a type 1 diabetes autoantigen, an alopecia areata autoantigen, a systemic lupus erythematosus autoantigen, a Behçet's disease autoantigen, a Sjögren's syndrome autoantigen, a rheumatoid arthritis autoantigen, a Grave's disease autoantigen, an antiphospholipid antibody syndrome autoantigen, a multiple sclerosis autoantigen, an irritable bowel disease autoantigen, a Crohn's disease autoantigen, an ulcerative colitis autoantigen and a dermatomyositis autoantigen.

30. A method for treating an autoimmune disease, comprising:
a) providing;
i) a B Cell Receptor Adaptor Immunoglobulin M (BCRAM) complex comprising an autoantibody-autoantigen complex;
ii) a cell comprising a B cell receptor (BCR) and a Toll-Like receptor (TLR), wherein said cell is within a patient exhibiting at least one symptom of an autoimmune disease;
b) administering said BCRAM complex to said patient under conditions such that said at least one symptom is reduced.

31. The method of claim 30, wherein said BCRAM complex comprises a human IgG variable domain.

32. The method of claim 30, wherein said autoantigen is selected from at least one of the group consisting of a synthetic autoantigen, a derivatized autoantigen and a conjugated autoantigen.

33. The method of claim 30, wherein said autoantigen is an autoimmune disease autoantigen.

34. The method of claim 33, wherein said autoimmune disease autoantigen is selected from at least one of the group consisting of a type 1 diabetes autoantigen, an alopecia areata autoantigen, a systemic lupus erythematosus autoantigen, a Behçet's disease autoantigen, a Sjögren's syndrome autoantigen, a rheumatoid arthritis autoantigen, a Grave's disease autoantigen, an antiphospholipid antibody syndrome autoantigen, a multiple sclerosis autoantigen, an irritable bowel disease autoantigen, a Crohn's disease autoantigen, an ulcerative colitis autoantigen and a dermatomyositis autoantigen.
